SEQUENCE LISTING (2 sequences)

SEQ ID NO: 1 - DNA, 21 bp, Artificial Sequence
  siRNA scr sense
cuuacgcugaguacuucgatt

SEQ ID NO: 2 - DNA, 21 bp, Artificial Sequence
  siRNA scr antisense
ucgaaguacucagcguaagtt

(19) United States
(12) Patent Application Publication
(10) Pub. No.: US 2013/0011336 A1
(43) Pub. Date: Jan. 10, 2013

(54) IMAGING AGENTS OF FIBROTIC DISEASES

(75) Inventors: Yoshiro Niitsu, Sapporo-shi (JP); Lei Yu, Carlsbad, CA (US); Gang Zhao, Vista, CA (US); Sang Van, San Diego, CA (US); Xinghe Wang, San Diego, CA (US); Jian Liu, San Diego, CA (US); Sanjib Kumar Das, Carlsbad, CA (US); Yasunobu Tanaka, Osaka (JP); Keiko Kajiwara, Osaka (JP); Hirokazu Takahashi, Osaka (JP); Miyono Miyazaki, Hokkaido (JP)

(73) Assignee: NITTO DENKO CORPORATION, Osaka (JP)

(21) Appl. No.: 13/063,193

(22) PCT Filed: Sep. 11, 2009
(86) PCT No.: PCT/JP2009/004521

§ 371 Date: May 31, 2012

Related U.S. Application Data
(60) Provisional application No. 61/096,488, filed on Sep. 12, 2008.

(30) Foreign Application Priority Data
Aug. 7, 2009 (JP) 2009-184806

(51) Int. Cl. A61K 49/00; A61K 49/04; A61K 49/10; G01N 21/62; A61K 51/06; A61K 49/22; A61P 1/16; C08G 69/48; C07C 403/08; A61K 51/04

(52) U.S. Cl. 424/1.89; 568/824; 424/9.4; 424/9.3; 424/9.6; 424/1.65; 424/9.5; 424/9.1; 424/1.85; 525/420; 424/1.81; 435/29

(57) ABSTRACT

Agents and methods for imaging a cell and/or a portion of tissue characterized by fibrosis, as well as to agents and methods for determining and/or diagnosing fibrotic diseases are disclosed herein. Also disclosed herein are polymer conjugates that can include a detectable label, a retinoid and a polymer. The polymer conjugates can be used to image a portion of tissue, deliver a detectable label to a portion of tissue or a cell and/or diagnosis a condition or disease.

### CROSS-REFERENCE TO RELATED APPLICATIONS

This application claims benefit of U.S. Provisional Patent Application Ser. No. 61/096,488, filed Sep. 12, 2008, and Japanese Patent Application No. 2009-184806, filed Aug. 7, 2009, the contents of which are hereby incorporated herein by reference in its entirety.

### TECHNICAL FIELD

Disclosed herein are compositions and methods related to the fields of organic chemistry, pharmaceutical chemistry, biochemistry, molecular biology and medicine. In particular, embodiments disclosed herein relate to agents and methods for imaging a cell and/or a portion of tissue, for example, a cell and/or tissue characterized by fibrosis, as well as to agents and methods for determining and/or diagnosing fibrotic diseases.

### BACKGROUND ART

Fibrosis, or the development of excess fibrous connective tissue within the body, has been associated with a number of diseases and disorders such as hepatic fibrosis, pancreatic fibrosis, vocal cord scarring, and numerous forms of cancer. Fibrotic diseases are a group of diseases characterized by such fibrosis, which may occur in various tissues such as liver. For instance, hepatic fibrosis, which is one of fibrotic disease, is caused, e.g., by hepatic stellate cells (HSC) being activated as a result of wound healing after tissue injury inside the liver due to viral hepatic disease caused by hepatitis B or C virus, nonalcoholic steatohepatitis, malnutrition-related diabetes, parasites, infectious diseases such as tuberculosis or syphilis, intrahepatic congestion due to heart disease, or a disorder in the passage of bile, etc., which in turn excessively produce and secrete extracellular matrix (ECM) such as a plurality of types of collagen molecules and fibronectin which is deposited on interstitial tissue. The final stage of hepatic fibrosis is hepatic cirrhosis which may cause hepatic failure, hepatocellular carcinoma, etc.

Various approaches have been taken in an attempt to inhibit fibrosis in an organ or tissue. One approach can be to inhibit the activation of one or more stellate cells, wherein activation of such cells is characterized by an increased production of extra-cellular matrix (ECM). Other approaches may relate to inhibiting the production of collagen, such as by promoting collagen degradation or controlling collagen metabolism. However, a method for recovering fibrotic tissue still does not exist, and when a large part of a tissue has been substituted with fibrotic tissue to the extent that the affected tissue cannot normally function, there are actually no options other than transplantation for recovering such a tissue. Therefore, it is crucial to detect fibrotic disease in an early stage, and provide an anti-fibrotic treatment.

Diagnosis of fibrotic diseases is usually done by a biopsy of tissue suspected of fibrosis. However, since the biopsy is a highly invasive technique which may cause complications such as infection, hemorrhage, pain, and injury of other tissue, various studies relating to non-invasive diagnostic methods of fibrotic diseases are done. For instance, an attempt to correlate analytic values of diffusion weighted MR images with the presence of cirrhosis (Aube et al., J Radiol. 2004; 85(3):301-6), an attempt to determine the presence of cirrhosis by detecting characteristic features thereof such as morphological changes of the liver by CT, MRI or ultrasonography (Kudo et al., Inter-virology. 2008; 51 Suppl 1:17-26), and an attempt to determine the presence of cirrhosis by biochemical indicators such as hyaluronate and prothrombin index (Oberti et al., Gastroenterology. 1997; 113(5):1609-16) are reported. However, none of such methods are satisfiable, and development of further diagnostic techniques is needed.

In addition, JP, A, 2009-518372 discloses the synthesis of retinoyl-PEG(12)-Lys-OH as a diagnostic contrast agent suitable for diagnostic imaging of fibrosis, but it does not describe that this substance was really useful as contrast agents.

### SUMMARY OF INVENTION

### Technical Problem

The present invention mainly aims to provide an imaging agent of fibrotic diseases, an imaging method of fibrotic disease using said imaging agent, a diagnostic agent of fibrotic disease comprising said imaging agent, a diagnostic method of fibrotic disease using said diagnostic agent, etc.

### Solution to Problem

In studying a novel diagnostic method of fibrotic diseases, the inventors have found that fibrotic diseases may be non-invasively detected in vivo by administering an imaging agent that comprises a retinoid and a detectable label to achieve the present invention. Although it was known that a carrier comprising vitamin A delivers drug to hepatic stellate cells (WO 2006/068232), it was not known at all that fibrotic diseases may be non-invasively detected in vivo by an imaging agent that comprises a retinoid and a detectable label.

The present invention relates to the following:

(1) An imaging agent of a cell and/or tissue characterized by fibrosis comprising a retinoid and a detectable label.

(2) The imaging agent of (1), wherein the retinoid comprises retinol.

(3) The imaging agent of (1) or (2), wherein the imaging agent is for in vivo imaging.

(4) The imaging agent of any one of (1)-(3), wherein the imaging agent is for fibrotic disease imaging.

(5) The imaging agent of any one of (1)-(4), comprising a polymer conjugate comprising at least one recurring unit selected from Formulae (I), (II), (III) and (IV):

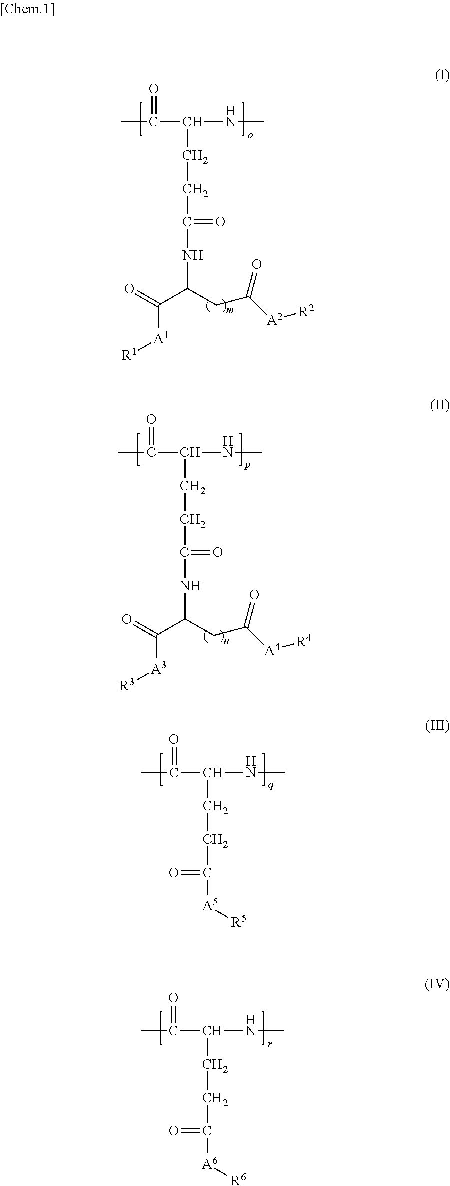

wherein:

m is independently 1 or 2;

n is independently 1 or 2;

A¹ and A² are each independently oxygen or NR⁷;

A³ and A⁴ are each independently oxygen or NR⁸;

A⁵ and A⁶ are each independently oxygen or NR⁹;

R¹, R², R³, R⁴, R⁵, and R⁶ are each independently selected from the group consisting of optionally substituted C₁₋₁₀ alkyl, optionally substituted C₆₋₂₀ aryl, ammonium, alkali metal, a retinoid and a group that comprises a detectable label;

R⁷, R⁸ and R⁹ are each independently hydrogen or C₁₋₄ alkyl;

o, p, q, and r are each independently 0, 1 or greater, wherein the sum of o, p, q, and r is 2 or greater; and

provided that at least one of R¹, R², R³, R⁴, R⁵, and R⁶ is a group that comprises a detectable label and at least one of R¹, R², R³, R⁴, R⁵, and R⁶ is a retinoid.

(6) A polymer conjugate comprising at least one recurring unit selected from Formulae (I), (II), (III) and (IV):

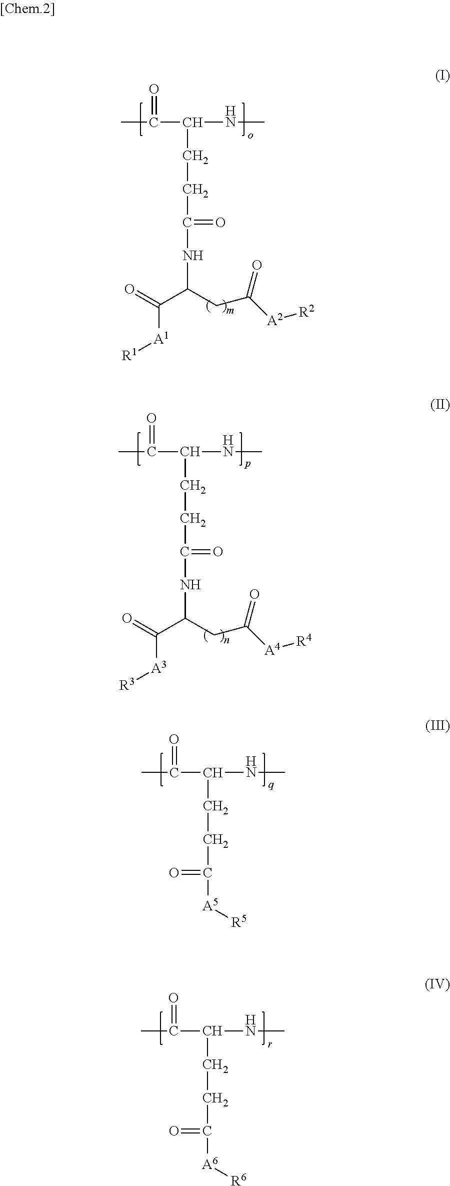

wherein:

m is independently 1 or 2;

n is independently 1 or 2;

A¹ and A² are each independently oxygen or NR⁷;

A³ and A⁴ are each independently oxygen or NR⁸;

A⁵ and A⁶ are each independently oxygen or NR⁹;

R¹, R², R³, R⁴, R⁵, and R⁶ are each independently selected from the group consisting of optionally substituted C₁₋₁₀ alkyl, optionally substituted C₆₋₂₀ aryl, ammonium, alkali metal, a retinoid and a group that comprises a detectable label;

R⁷, R⁸ and R⁹ are each independently hydrogen or C₁₋₄ alkyl;

o, p, q, and r are each independently 0, 1 or greater, wherein the sum of o, p, q, and r is 2 or greater; and
provided that at least one of R¹, R², R³, R⁴, R⁵, and R⁶ is a group that comprises a detectable label and at least one of R¹, R², R³, R⁴, R⁵, and R⁶ is a retinoid.

(7) The polymer conjugate of (6), wherein the polymer further comprises at least one recurring unit of Formula (V):

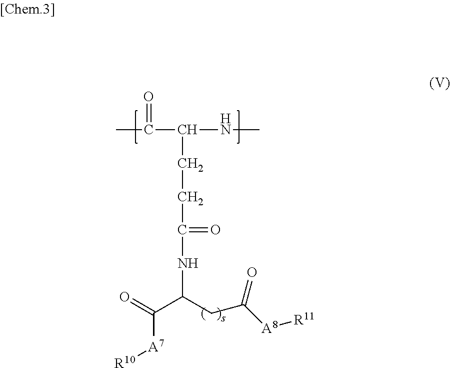

wherein:

s is independently 1 or 2;

A⁷ and A⁸ are each independently oxygen or NR¹²;

R¹² is hydrogen or C₁₋₄ alkyl;

R¹⁰ and R¹¹ are each independently selected from the group consisting of optionally substituted C₁₋₁₀ alkyl, optionally substituted C₆₋₂₀ aryl, ammonium and alkali metal.

(8) The polymer conjugate of (6) or (7), wherein the polymer further comprises at least one recurring unit of Formula (VI):

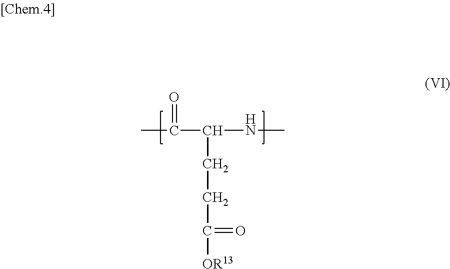

wherein R¹³ is hydrogen, ammonium, or an alkali metal.

(9) The polymer conjugate of any one of (6)-(8), wherein the detectable label comprises a metal selected from the group consisting of Gd(III), Yttrium-88 and Indium-111.

(10) The polymer conjugate of any one of (6)-(9), wherein the detectable label comprises a ligand selected from the group consisting of: diethylenetriaminepentacetic acid (DTPA), tetraazacyclododecane-1,4,7,10-tetraacetic acid (DOTA), (1,2-ethanediyldinitrilo)tetraacetate (EDTA), ethylenediamine, 2,2′-bipyridine (bipy), 1,10-phenanthroline (phen), 1,2-bis(diphenylphosphino)ethane (DPPE), 2,4-pentanedione (acac), and ethanedioate (ox).

(11) The polymer conjugate of any one of (6)-(10), wherein the detectable label comprises a ligand selected from the group consisting of: diethylenetriaminepentacetic acid (DTPA) and tetraazacyclododecane-1,4,7,10-tetraacetic acid (DOTA).

(12) The polymer conjugate of any of (6)-(11), wherein the detectable label is a para-magnetic metal chelate.

(13) The polymer conjugate of (12), wherein the paramagnetic metal chelate comprises

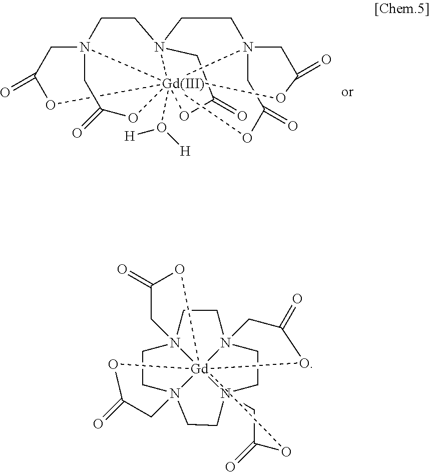

(14) The polymer conjugate of any of (6)-(11), wherein the detectable label is a dye.

(15) The polymer conjugate of (14), wherein the dye comprises Texas Red.

(16) The polymer conjugate of any one of (6)-(15), wherein m is 1.

(17) The polymer conjugate of any one of (6)-(15), wherein m is 2.

(18) The polymer conjugate of any one of (6)-(17), wherein n is 1.

(19) The polymer conjugate of any one of (6)-(17), wherein n is 2.

(20) The polymer conjugate of any one of claims 7)-(19), wherein s is 1.

(21) The polymer conjugate of any one of claims 7)-(19), wherein s is 2.

(22) A method of making the polymer conjugate of any one of (6)-(21) comprising

dissolving or partially dissolving a polymeric reactant comprising at least one of a recurring unit of Formula (VII) and a recurring unit of Formula (VIII) in a solvent to form a dissolved or partially dissolved polymeric reactant;

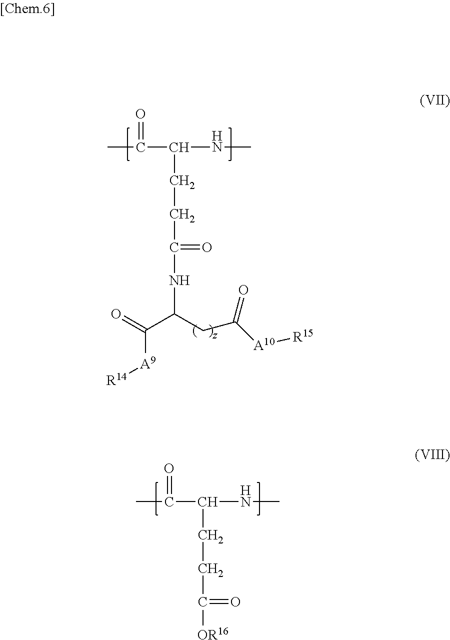

wherein:
z is 1 or 2;
A⁹ and A¹⁰ are oxygen; and
R¹⁴, R¹⁵ and R¹⁶ are each independently selected from the group consisting of hydrogen, ammonium, and an alkali metal; and
reacting the dissolved or partially dissolved polymeric reactant with a second reactant,
wherein the second reactant comprises the group comprising the detectable label or the retinoid; and
adding a third reactant, wherein the third reactant comprises the group comprising the detectable label, a ligand or the retinoid; provided that if the second reactant comprises the group comprising the detectable label or the ligand then the third reactant comprises the retinoid and if the second reactant comprises the retinoid then the third reactant comprises the group that comprises the detectable label or the ligand.

(23) The method of (22), wherein the second reactant comprises the retinoid.

(24) The method of (22) or (23), wherein the third reactant comprises the group that comprises the detectable label.

(25) The method of (22) or (23), wherein the third reactant comprises the ligand.

(26) The method of (25), wherein the ligand is selected from the group consisting of: diethylenetriaminepentacetic acid (DTPA), tetraazacyclododecane-1,4,7,10-tetraacetic acid (DOTA), (1,2-ethanediyldinitrilo)tetraacetate (EDTA), ethylenediamine, 2,2′-bipyridine (bipy), 1,10-phenanthroline (phen), 1,2-bis(diphenylphosphino)ethane (DPPE), 2,4-pentanedione (acac), and ethanedioate (ox).

(27) The method of any one of (22)-(26), further comprising adding a fourth reactant, wherein the fourth reactant comprises a metal.

(28) The method of (27), wherein the metal is selected from the group consisting of Gd(III), Yttrium-88 and Indium-111.

(29) A diagnostic agent of fibrotic disease comprising the imaging agent of any one of (1)-(5) and/or the polymer conjugate of any one of (6)-(21).

(30) A composition comprising the imaging agent of any one of (1)-(5) and/or the polymer conjugate of any one of (6)-(21) and/or the diagnostic agent of (29), and at least one selected from a pharmaceutically acceptable excipient, a carrier, and a diluent.

(31) A method of delivering a detectable label to a portion of tissue comprising contacting the portion of tissue or a cell with at least one imaging agent of any one of (1)-(5) and/or the polymer conjugate of any one of (6)-(21) and/or the diagnostic agent of (29), and/or the composition of (30).

(32) A method of imaging a portion of tissue comprising contacting the portion of tissue or a cell with at least one imaging agent of any one of (1)-(5) and/or the polymer conjugate of any one of (6)-(21) or the composition of (30).

(33) A method of diagnosing a disease or condition comprising contacting a portion of tissue or a cell with at least one polymer conjugate of any one of (6)-(21), and/or the diagnostic agent of (29), and/or the composition of (30).

(34) The method of any one of (31)-(33), wherein the tissue is fibrous tissue.

(35) A method for imaging a fibrotic disease comprising a step of administering an effective amount of the imaging agent of any one of (1)-(5), the polymer conjugate of any one of (6)-(21) and/or the composition of (30) to a subject in need thereof, and a step of detecting the label contained in the administered imaging agent, polymer conjugate or composition.

(36) A method for determining fibrotic disease comprising a step of comparing a signal intensity and/or a signal distribution of a label detected from a subject to which the imaging agent of any one of (1)-(5), and/or the polymer conjugate of any one of (6)-(21), and/or the diagnostic agent of (29), and/or the composition of (30) are/is administered, with a reference signal intensity and/or a reference signal distribution.

(37) A method of monitoring fibrotic disease comprising a step of comparing a signal intensity and/or a signal distribution of a label detected at a first time point from a subject to which the imaging agent of any one of (1)-(5), and/or the polymer conjugate of any one of (6)-(21), and/or the diagnostic agent of (29), and/or the composition of (30) are/is administered, with a signal intensity and/or a signal distribution of a label detected at a second time point that is later than the first time point from said subject.

A method for determining an effect of a fibrotic disease treatment comprising a step of comparing a signal intensity and/or a signal distribution of a label detected at a first time point from a subject to which the imaging agent of any one of (1)-(5), and/or the polymer conjugate of any one of (6)-(21), and/or the diagnostic agent of (29), and/or the composition of (30) are/is administered, with a signal intensity and/or a signal distribution of a label detected at a second time point that is later than the first time point from said subject, wherein the first time point is before the subject receives the fibrotic disease treatment, and the second time point is after the subject has received the fibrotic disease treatment, or alternatively, the first time point is after the subject received a first fibrotic disease treatment, and the second time point is after the subject received a second fibrotic disease treatment that is made after the first fibrotic disease treatment.

Some embodiments described herein relate to a polymer conjugate that can include at least one recurring unit selected from Formulae (I), (II), (III) and (IV) as set forth herein.

Other embodiments described herein related to a method of delivering a detectable label to a portion of tissue or a cell that can include contacting the portion of tissue or the cell with at least one of the polymer conjugates described herein.

Still other embodiments described herein relate to a method of imaging a portion of tissue or a cell that can include contacting the portion of tissue or the cell with at least one of the polymer conjugates described herein.

Yet still other embodiments described herein related to a method of diagnosing a disease or condition such as a disease or condition characterized by fibrosis that can include contacting a portion of tissues with at least one of the polymer conjugates described herein.

Some embodiments described herein related to use of at least one of the polymer conjugates described herein for delivering a detectable label to a portion of tissue or a cell.

Other embodiments described herein relate to use of at least one of the polymer conjugates described herein for imaging a portion of tissue or a cell.

Still other embodiments described herein related to use of at least one of the polymer conjugates described herein for diagnosing a disease or condition such as a disease or condition characterized by fibrosis.

Some embodiments described herein related to a polymer conjugate described herein for delivering a detectable label to a portion of tissue or a cell.

Other embodiments described herein relate to a polymer conjugate described herein for imaging a portion of tissue or a cell.

Still other embodiments described herein related to a polymer conjugate described herein for diagnosing a disease or condition such as a disease or condition characterized by fibrosis.

### Advantageous Effects of Invention

Although the accurate mechanism of the imaging agent of the present invention has not been completely elucidated, it is surmised that retinoid functions as targeting agent for alpha-SMA (smooth muscle actin)-positive ECM (extracellular matrix)-producing cells such as activated stellate cells, and makes it possible to detect said cells by delivering thereto a labeling substance.

Since the imaging agent of the present invention allows to nondestructively, preferably noninvasively detect fibrotic diseases in vivo, the risk of complications due to conventional biopsy is eliminated, and therefore, it is possible to considerably reduce the burden of a subject to be examined. In addition, since this may broaden the range of subjects to be examined, early detection of fibrotic diseases is facilitated, which allows an effective slowing of progression of said diseases. Thus, the present invention makes a considerably large contribution in human and veterinary medicines.

Furthermore, the imaging agent of the present invention allows to nondestructively, preferably noninvasively observe a fibrotic disease in vivo over time in a same individual, which leads to evaluate the treatment of said disease with high accuracy.

These and other embodiments are described in greater detail below.

### BRIEF DESCRIPTION OF DRAWINGS

FIG. 1 illustrates a reaction scheme for the preparation of a polymer conjugate that includes a retinoid, polyglutamate and tetraazacyclododecane-1,4,7,10-tetraacetic acid (DOTA), Retinoid-PGA-DOTA.

FIG. 2 illustrates a reaction scheme for the preparation of a polymer conjugate that includes a retinoid, polyglutamate and diethylenetriaminepentacetic acid (DTPA), Retinoid-PGA-DTPA.

FIG. 3 illustrates a reaction scheme for the preparation of a polymer conjugate that includes a retinoid, poly(L-gamma-glutamylglutamine) and tetraazacyclododecane-1,4,7,10-tetraacetic acid (DOTA), Retinoid-PGGA-DOTA.

FIG. 4 illustrates a reaction scheme for the preparation of a polymer conjugate that includes a retinoid, poly(L-gamma-glutamylglutamine) and diethylenetriaminepentacetic acid (DTPA), Retinoid-PGGA-DTPA.

FIG. 5 illustrates a reaction scheme for the preparation of a polymer conjugate that includes a retinoid, polyglutamate and tetraazacyclododecane-1,4,7,10-tetraacetic acid (DOTA)Gd(III), Retinoid-PGA-[(DOTA)Gd(III)].

FIG. 6 illustrates a reaction scheme for the preparation of a polymer conjugate that includes a retinoid, polyglutamate and diethylenetriaminepentacetic acid (DTPA)Gd(III), Retinoid-PGA-[(DTPA)Gd(III)].

FIG. 7 illustrates a reaction scheme for the preparation of a polymer conjugate that includes a retinoid, poly(L-gamma-glutamylglutamine) and tetraazacyclododecane-1,4,7,10-tetraacetic acid (DOTA)Gd(III), Retinoid-PGGA-[(DOTA)Gd(III)].

FIG. 8 illustrates a reaction scheme for the preparation of a polymer conjugate that includes a retinoid, poly(L-gamma-glutamylglutamine) and diethylenetriaminepentacetic acid (DTPA)Gd(III), Retinoid-PGGA-[(DTPA)Gd(III)].

FIG. 9 is a graph illustrating the cell uptake of Texas Red (TR)-polyglutamate (PGA)-Retinoid, TR-PGA-Retinoid, as compared to Texas Red (TR)-polyglutamate (PGA)-cholesterol, Texas Red-PGA-cholesterol.

FIG. 10 shows MRI images of the fibrosis DMA rat models over time.

FIG. 11 is a plot of relative optical density of Gd(III) of PGGA-[(DPTA)Gd(III)] and Retinoid-PGGA-[(DPTA)Gd(III)] in the liver of DMA rat models over time. The amount of Gd(III) of retinoid-PGGA-[(DPTA)Gd(III)] detected in the liver of the rats was much higher than the amount of Gd(III) of PGGA-[(DPTA)Gd(III)] detected in the liver of the fibrosis DMA rat models.

FIG. 12 illustrates the intensity and the distribution of fluorescent signal of cirrhotic mouse (left) and normal mouse (right) 5 min (upper) and 90 min (lower) after administration of the imaging agent. The intensity of fluorescent signal is indicated by average radiance (Avg Radiance, p/s/cm²/sr).

FIG. 13 is a graph showing over time change of the intensity of fluorescent signal at the liver (left) and the intestine (right) of cirrhotic mouse (filled circle) and normal mouse (filled square) from 5 to 90 min after administration of the imaging agent.

FIG. 14 illustrates the localization of fluorescent signal in liver tissue of cirrhotic mouse (left) and normal mouse (right) 90 min after administration of an image agent with (upper panels) or without (lower panels) VA.

FIG. 15 is a graph showing the ratio of Cy™5.5/FITC double positive area to total Cy™5.5-positive area in the liver of cirrhotic mouse and normal mouse 90 min after administration of an image agent (mean of 8 random fields).

FIG. 16 is a graph showing the ratio of the number of Cy™5.5/FITC double positive cells to the total number of Cy™5.5-positive cells in the liver of cirrhotic mouse and normal mouse 90 min after administration of an image agent (mean of 8 random fields).

### DESCRIPTION OF EMBODIMENTS

Early diagnosis of fibrosis can provide a greater opportunity to find an appropriate treatment. Currently, the only clinically available approved method for diagnosing fibrosis is a biopsy. However, a biopsy requires the removal of tissue for examination. A non-invasive method would minimize the need to remove tissue and the risks associated with having a foreign object inserted into the subject. Such a non-invasive method would address an unmet option for diagnosing fibrosis.

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there are a plurality of definitions for a term herein, those in this section prevail unless stated otherwise.

The term “ester” is used herein in its ordinary sense, and thus includes a chemical moiety with formula —(R)n-COOR′, where R and R′ are independently selected from the group consisting of alkyl, cycloalkyl, aryl, heteroaryl (bonded through a ring carbon) and heteroalicyclic (bonded through a ring carbon or heteroatom), and where n is 0 or 1.

The term “amide” is used herein in its ordinary sense, and thus includes a chemical moiety with formula —(R)n-C(O)NHR′ or —(R)n-NHC(O)R′, where R and R′ are independently selected from the group consisting of alkyl, cycloalkyl, aryl, heteroaryl (bonded through a ring carbon) and heteroalicyclic (bonded through a ring carbon or heteroatom), and where n is 0 or 1. An amide may be included in an amino acid or a peptide molecule attached to a drug molecule as described herein, thereby forming a prodrug.

Any amine, hydroxy, or carboxyl side chain on the compounds disclosed herein can be esterified or amidified. The procedures and specific groups to be used to achieve this end are known to those of skill in the art and can readily be found in reference sources such as Greene and Wuts, Protective Groups in Organic Synthesis, 3rd Ed., John Wiley & Sons, New York, N.Y., 1999, which is incorporated herein in its entirety.

As used herein, “alkyl” refers to a straight or branched hydrocarbon chain that comprises a fully saturated (no double or triple bonds) hydrocarbon group. The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 10 carbon atoms. The alkyl group could also be a lower alkyl having 1 to 5 carbon atoms. The alkyl group of the compounds may be designated as “C₁-C₄ alkyl” or similar designations. By way of example only, “C₁-C₄ alkyl” indicates that there are one to four carbon atoms in the alkyl chain, i.e., the alkyl chain is selected from methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl, and the like.

The alkyl group may be substituted or unsubstituted. When substituted, the substituent group(s) is(are) one or more group(s) individually and independently selected from alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxyl, alkoxy, aryloxy, acyl, ester, mercapto, alkylthio, arylthio, cyano, halogen, carbonyl, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl (e.g., mono-, di- and tri-haloalkyl), haloalkoxy (e.g., mono-, di- and tri-haloalkoxy), trihalomethanesulfonyl, trihalomethanesulfonamido, and amino, including mono- and di-substituted amino groups, and the protected derivatives thereof. Wherever a substituent is described as being “optionally substituted” that substitutent may be substituted with one of the above substituents.

As used herein, “aryl” refers to a carbocyclic (all carbon) monocyclic or multicyclic aromatic ring system that has a fully delocalized pi-electron system. Examples of aryl groups include, but are not limited to, benzene, naphthalene and azulene. An aryl group of this invention may be substituted or unsubstituted. When substituted, hydrogen atoms are replaced by substituent group(s) that is(are) one or more group(s) independently selected from alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxy, alkoxy, aryloxy, acyl, ester, mercapto, cyano, halogen, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl (e.g., mono-, di- and tri-haloalkyl), haloalkoxy (e.g., mono-, di- and tri-haloalkoxy), trihalomethanesulfonyl, trihalomethanesulfonamido, and amino, including mono- and di-substituted amino groups, and the protected derivatives thereof, unless the substituent groups are otherwise indicated.

A “paramagnetic metal chelate” is a complex wherein a ligand is bound to a para-magnetic metal ion. Examples include, but are not limited to, 1,4,7,10-Tetraazacyclododecane-1,4,7,10-tetraacetic acid (DOTA)-Gd(III), DOTA-Yttrium-88, DOTA-Indium-111, diethylenetriaminepentaacetic acid (DTPA)-Gd(III), DTPA-yttrium-88, DTPA-Indium-111.

A “retinoid” is a member of the class of compounds consisting of four isoprenoid units joined in a head-to-tail manner, see G. P. Moss, “Biochemical Nomenclature and Related Documents,” 2nd Ed. Portland Press, pp. 247-251 (1992). “Vitamin A” is the generic descriptor for retinoids exhibiting qualitatively the biological activity of retinol. As used herein, retinoid refers to natural and synthetic retinoids including first generation, second generation, and third generation retinoids. Examples of naturally occurring retinoids include, but are not limited to, (1) 11-cis-retinal, (2) all-trans retinol, (3) retinyl palmitate, (4) all-trans retinoic acid, and (5) 13-cis-retinoic acids. Furthermore, the term “retinoid” encompasses retinols, retinals, and retinoic acids.

“Fibrosis” is used herein in its ordinary sense and refers to the development of fibrous scar-like connective tissue in an organ or tissue as part of a reparative or reactive process. “Abnormal fibrosis” refers to the development of fibrous scar-like connective tissue in an organ or tissue to an extent that it impairs the function of the organ or tissue. “Fibrotic disease” refers herein to any diseases characterized by a fibrosis of tissue, and includes, but not limited to, e.g., hepatic fibrosis, hepatic cirrhosis, vocal cord scarring, vocal cord mucosal fibrosis, laryngeal fibrosis, pulmonary fibrosis, myelofibrosis, myocardial infarction, and fibrosis of myocardium after myocardial infarction. In the purpose of the present invention, fibrotic diseases typically are those in which alpha-SMA-positive extracellular matrix-producing cells, such as hepatic stellate cells are involved.

As used herein, “linker” and “linking group” refer to one or more atoms that connect one chemical moiety to another chemical moiety. Examples of linking groups include relatively low molecular weight groups such as amide, ester, carbonate and ether, as well as higher molecular weight linking groups such as polyethylene glycol (PEG).

It is understood that, in any compound described herein having one or more chiral centers, if an absolute stereochemistry is not expressly indicated, then each center may independently be of R-configuration or S-configuration or a mixture thereof. Thus, the compounds provided herein may be enantiomerically pure or be stereoisomeric mixtures. In addition it is understood that, in any compound described herein having one or more double bond(s) generating geometrical isomers that can be defined as E or Z each double bond may independently be E or Z a mixture thereof. Likewise, all tautomeric forms are also intended to be included.

As used herein, the abbreviations for any protective groups, amino acids and other compounds are, unless indicated otherwise, in accord with their common usage, recognized abbreviations, or the IUPAC-IUP Commission on Biochemical Nomenclature (See, Biochem. 11:942-944 (1972)).

One aspect of the present invention relates to an imaging agent of fibrotic diseases comprising a retinoid and a detectable label.

Retinoid in the present invention can target alpha-SMA-positive ECM-producing cells such as activated stellate cells which are involved in fibrotic disease. Although the mechanism by which a retinoid targets alpha-SMA-positive ECM-producing cells has not been completely elucidated, it is surmised, e.g., that retinoid specifically bound to RBP (retinol binding protein) is bound to and/or uptaken into said cells via a certain type of receptor on the surface thereof.

Retinoid that may be used in the present invention includes, but not limited to, retinoid derivatives such as retinol, retinal, retinoic acid, an ester of retinol and a fatty acid, an ester of an aliphatic alcohol and retinoic acid, etretinate, tretinoin, isotretinoin, adapalene, acitretine, tazarotene, and retinol palmitate, and vitamin A analogues such as fenretinide (4-HPR), and bexarotene. Retinoid also includes rexinoids, i.e., retinoid compounds which are selective for retinoid X receptors (RXR), such as bexarotene.

Among them, retinol, retinal, retinoic acid, an ester of retinol and a fatty acid (e.g. retinyl acetate, retinyl palmitate, retinyl stearate, and retinyl laurate) and an ester of an aliphatic alcohol and retinoic acid (e.g. ethyl retinoate) are preferable from the viewpoint of efficiency of specific targeting of said ECM-producing cells. In another embodiment, retinoid in the present invention does not include retinoic acid and/or retinoic acid derivatives. Thus, preferable retinoid in these embodiments includes retinol, retinal, an ester of retinol and a fatty acid, etc.

For the purpose of the present invention, retinoid also includes all retinoid isomers, such as cis/trans isomers. Specific examples of such isomers include, but not limited to, e.g., all-trans retinol, all-trans retinoic acid, 11-cis-retinal and 13-cis-retinoic. The retinoid may be substituted with one or more substituents. The retinoid in the present invention includes a retinoid in an isolated state as well as in a solution or mixture state with a medium that can dissolve or retain the retinoid.

A detectable label (or simply referred to as “label”) in the present invention includes any labels that can be detected by any existing detection means. A detection means includes, but not limited to, e.g., naked eye, optical examination apparatus (such as optical microscope, fluorescent microscope, phase contrast microscope, in vivo imaging apparatus), X-ray apparatus (such as plain X-ray apparatus, CT (computed tomography) apparatus), MRI (magnetic resonance imaging) apparatus, nuclear medicine apparatus (such as scintigraphic apparatus, PET (positron emission tomography) apparatus, SPECT (single photon emission computed tomography) apparatus), ultrasonographic apparatus and thermographic apparatus. Labels suitable for each detection means are known to a person skilled in the art, and described, e.g., in Lecchi et al., Q J Nucl Med Mol. Imaging. 2007; 51(2):111-26.

Label suitable for detection by naked eye and optical examination apparatus includes, e.g., various fluorescent labels and luminescent labels. Specific fluorescent label which may be used, includes, but not limited to, e.g., Cy™ series (such as Cy™2, 3, 5, 5.5, 7), DyLight™ series (such as DyLight™ 405, 488, 549, 594, 633, 649, 680, 750, 800), Alexa Fluor® series (such as Alexa Fluor® 405, 488, 549, 594, 633, 647, 680, 750), HiLyte Fluor™ series (such as HiLyte Fluor™ 488, 555, 647, 680, 750), ATTO series (such as ATTO 488, 550, 633, 647N, 655, 740), FAM, FITC, Texas-Red, GFP, RFP and Qdot. In the present invention, fluorescent labels suitable for in vivo imaging are, e.g., those emit a fluorescence of wavelength that are highly transmissive through living body and less susceptible to autonomous fluorescence, such as a fluorescence of near-infrared wavelength, or those exhibit strong fluorescent intensity. Such fluorescent labels include, but not limited to, e.g., Cy™ series, DyLight™ series, Alexa Fluor® series, HiLyte Fluor™ series, ATTO series, Texas-Red, GFP, RFP, Qdot and derivatives thereof.

Specific luminescent label which may be used, includes, but not limited to, e.g., luminol, luciferin, lucigenin and aequorin.

Suitable label for detection by X-ray apparatus includes, e.g., various contrast agents. Specific contrast agent which may be used, includes, but not limited to, e.g., iodine atom, iodine ions and iodine-containing compounds.

Suitable label for detection by MRI apparatus includes, e.g., various metal atoms, and compounds containing said metal atom(s), such as complexes of said metal atom(s). Specifically, one may use, but not limited to, e.g., gadolinium(III) (Gd(III)), yttrium-88 (⁸⁸Y), indium-111 (¹¹¹In), complexes of such metal atom(s) and a ligand such as diethylenetriaminepentacetic acid (DTPA), tetraazacyclododecane-1,4,7,10-tetraacetic acid (DOTA), (1,2-ethanediyldinitrilo)tetraacetate (EDTA), ethylenediamine, 2,2′-bipyridine (bipy), 1,10-phenanthroline (phen), 1,2-bis(diphenylphosphino)ethane (DPPE), 2,4-pentanedione (acac), and ethanedioate (ox), super-paramagnetic iron oxide (SPIO) and manganese oxide (MnO). A group that includes a paramagnetic metal chelate is one example of a suitable label for detection by MRI apparatus. In some embodiments, the paramagnetic metal chelate can include one of the following groups:

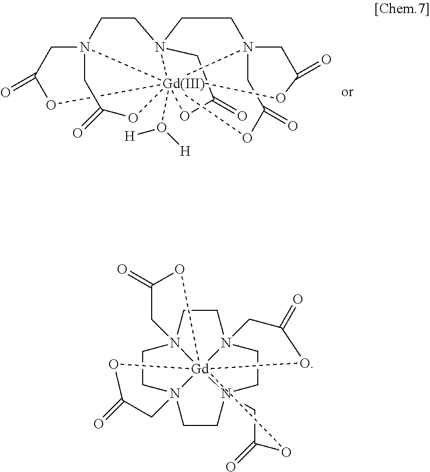

Suitable label for detection by nuclear medicine apparatus includes, e.g., various radioisotopes, and compounds containing said radioisotope(s), such as complexes of said radioisotope(s). Radioisotopes which may be used include, but not limited to, e.g., technetium-99m (^{99m}Tc), indium-111 (¹¹¹In), iodine-123 (¹²³I), iodine-124 (¹²³I), iodine-125 (¹²⁵I), iodine-131 (¹³¹I), thallium-201 (²⁰¹Tl), carbon-11 (¹¹C), nitrogen-13 (¹³N), oxygen-15 (¹⁵O), fluorine-18 (¹⁸F), copper-64 (⁶⁴Cu), gallium-67 (⁶⁷Ga), krypton-81m (^{81m}Kr), xenon-133 (¹³³Xe), strontium-89 (⁸⁹Sr) and yttrium-90 (⁹⁰Y). Compounds containing radioisotope include, but not limited to, e.g., ¹²³I-Imp, ^{99m}Tc-HMPAO, ^{99m}Tc-ECD, ^{99m}Tc-MDP, ^{99m}Tc-tetrofosmin, ^{99m}Tc-MIBI, ^{99m}TcO₄—, ^{99m}Tc-MAA, ^{99m}Tc-MAG3, ^{99m}Tc-DTPA, ^{99m}Tc-DMSA and ¹⁸F-FDG1.

Suitable label for detection by ultrasonographic apparatus which may be used, includes, but not limited to, e.g., nanoparticles and liposomes.

The imaging agent of the present invention may be only formed from the above-mentioned retinoid and label or may be formed by making these two components bind to or be enclosed in a carrier other than said components. Therefore, the imaging agent of the present invention may include a carrier other than retinoid and label. Such a carrier is not particularly limited, and any carrier known in the medicinal and pharmaceutical fields may be used, but those that can enclose retinoid or can bind thereto are preferable.

Examples of such a carrier include a lipid, for example, a phospholipid such as glycerophospholipid, a sphingolipid such as sphingomyelin, a sterol such as cholesterol, a vegetable oil such as soybean oil or poppy seed oil, a mineral oil, a lecithin such as egg-yolk lecithin, polymers, and carriers comprising a ligand that is not limited to a polymer, but the examples are not limited thereto. Among them, those that can form a liposome are preferable, for example, a natural phospholipid such as lecithin, a semisynthetic phospholipid such as dimyristoylphosphatidylcholine (DMPC), dipalmitoylphosphatidylcholine (DPPC), or distearoylphosphatidylcholine (DSPC), dio-leylphosphatidylethanolamine (DOPE), dilauroylphosphatidylcholin (DLPC), and cholesterol. Ligand may be a monodentate ligand or a multidentate ligand which can form a chelate.

A particularly preferred carrier is those that can avoid capture by the reticuloendothelial system, and examples thereof include cationic lipids such as N-(alpha-trimethylammonioacetyl)-didodecyl-D-glutamate chloride (TMAG), N,N′,N″,N′″-tetramethyl-N,N′,N″,N′″-tetrapalmitylspermine (TMTPS), 2,3-dioleyloxy-N-[2(sperminecarboxamido)ethyl]-N,N-dimethyl-1-propanaminium tri-fluoroacetate (DOSPA), N-[1-(2,3-dioleyloxy)propyl]-N,N,N-trimethylammonium chloride (DOTMA), dioctadecyldimethylammonium chloride (DODAC), didodecylammonium bromide (DDAB), 1,2-dioleyloxy-3-trimethylammoniopropane (DOTAP), 3b-[N—(N′,N-dimethylaminoethane)carbamoyl]cholesterol (DC-Chol), 1,2-dimyristoyloxypropyl-3-dimethylhydroxyethylammonium (DMRIE), and O,O′-ditetradecanoyl-N-(alpha-trimethylammonioacetyl)diethanolamine chloride (DC-6-14).

The binding of the retinoid and/or the label to the carrier of the present invention or the enclosing of the retinoid and/or the label therein is also possible by binding or enclosing the retinoid and/or the label to or in the carrier by a chemical and/or physical method. For example, the retinoid and/or the label can be directly attached to the carrier. In one embodiment, the retinoid and/or the label can be directly attached to the carrier through an oxygen, a sulfur, a nitrogen and/or a carbon atom of retinoid and/or the label. In other embodiments, retinoid and/or the label can further include a linker group. In an embodiment, the retinoid and/or the label can be attached to the carrier through a linker group. The linker group may be relatively small. For instance, the linker group may comprise an amine, an amide, an ether, an ester, a hydroxyl group, a carbonyl group, or a thioether group. Alternatively, the linker group may be relatively large. For instance, the linker group may comprise an alkyl group, an aryl group, an aryl(C₁₋₆ alkyl) group (e.g., phenyl-(CH₂)₁₋₄—), a heteroaryl group, or a heteroaryl(C₁₋₆ alkyl) group. In one embodiment, the linker can be —NH(CH₂)₁₋₄—NH—. In another embodiment, the linker can be —(CH₂)₁₋₄-aryl-NH—. As an example, the linker group can be attached in place of a hydrogen at a carbon of the retinoid and/or the label. The linker group can be added to the carrier using methods known to those skilled in the art.

Alternatively, the binding or enclosing of the retinoid and/or the label to or in the carrier can also be carried out by mixing the retinoid and/or the label and the carrier when preparing the present imaging agent. Alternatively, when the carrier comprises a ligand that is not limited to a polymer, it is possible to make a label detectable by magnetic resonance imaging or nuclear medicine examination be supported by said ligand.

The amount of retinoid in the imaging agent of the present invention may be preferably 1-10000 nanomol/microliter (nmol/microL), more preferably 10-1000 nmol/microL. The amount of label may be those known in the art, but may be appropriately increased or decreased considering various conditions such as the amount of retinoid and the nature of the carrier. The binding or enclosing of retinoid to or in the carrier may be carried out before the label is supported on the carrier, may be carried out at the same time as mixing the carrier, retinoid and the label, or may be carried out by mixing retinoid with a carrier on which a label is already supported. Therefore, the present invention also relates to a process for producing an imaging agent of fibrotic diseases, the process including a step of binding retinoid to any existing labeled formulation.

The form of the carrier of the present invention may be any form as long as a label can be carried to a target extracellular matrix-producing cell, and although not limited thereto, examples thereof include a macromolecular micelle, a liposome, an emulsion, microspheres, and nanospheres. When the carrier is in the form of liposome, the molar ratio of retinoid to liposome-forming lipid as a carrier is preferably 8:1 to 1:4, more preferably 4:1 to 1:2, considering the efficiency of binding or enclosing of retinoid to or in the carrier.

The carrier of the imaging agent of the present invention may contain the label within its interior, may attach it to the exterior, or may be mixed with the label, as long as the retinoid is present in such a configuration that it can function as targeting agent. “Function as targeting agent” referred to here means that the imaging agent containing retinoid reaches and/or is taken up by the target cell, i.e., an extracellular matrix-producing cell more rapidly, efficiently and/or in a larger quantity than with an imaging agent not containing retinoid, and this may easily be confirmed by, for example, adding the imaging agent of the present invention to a culture of target cells, and analyzing sites where the label is present after a predetermined period of time. Structurally, the above-mentioned requirements may be met, e.g., if retinoid is at least partially exposed on the exterior of the imaging agent at the latest by the time it reaches the target cell. Whether or not retinoid is at least partially exposed on the exterior of the imaging agent, may be evaluated by contacting the imaging agent with a substance that specifically binds to retinoid, such as retinol binding protein (RBP), and studying the binding thereof to the imaging agent. Exposing retinoid on the exterior of the imaging agent at least partially at the latest by the time it reaches the target cell, may be achieved, e.g., by adjusting the formulating ratio of the retinoid and the label and/or optionally the carrier.

In one embodiment, the carrier is a polymer which may comprise a recurring unit of Formula (V):

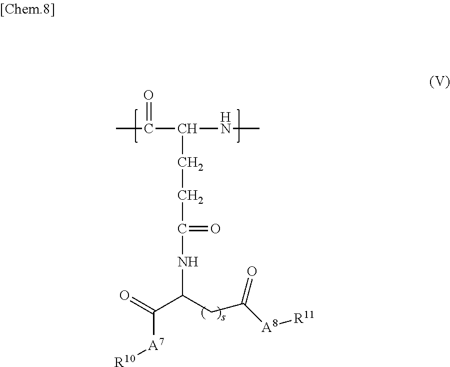

wherein: s can be independently 1 or 2; A⁷ and A⁸ can be each independently oxygen or NR¹²; R¹² can be hydrogen or C₁₋₄ alkyl; R¹⁰ and R¹¹ can be each independently selected from an optionally substituted C₁₋₁₀ alkyl, an optionally substituted C₆₋₂₀ aryl, ammonium and an alkali metal; and/or a recurring unit of Formula (VI):

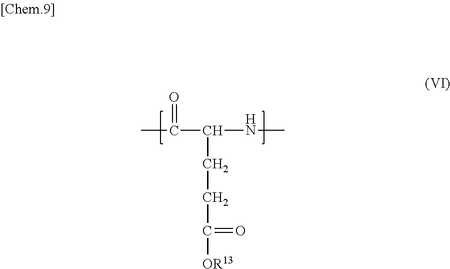

wherein R¹³ can be hydrogen, ammonium, or an alkali metal. When the R¹³ group is hydrogen, then the recurring unit of the Formula (VI) is a recurring unit of glutamic acid.

In this embodiment, at least one of R¹⁰, R¹¹ and R¹³ may be substituted with retinoid and/or a detectable label, so that both the retinoid and the detectable label are comprised in the imaging agent. In other words, retinoid and a detectable label may be bound to A⁷ and/or A⁸ of Formula (V) or to O atom bound to C═O of Formula (VI). However, retinoid and a detectable label may be bound to other part of the polymer. In a preferable embodiment, at least one of R¹⁰, R¹¹ and R¹³ is substituted with a detectable label and at least one of R¹⁰, R¹¹ and R¹³ is substituted with a retinoid.

Accordingly, the imaging agent of the present invention may comprise or consist of a polymer conjugate as defined below. In addition, one aspect of the present invention relates to said polymer conjugate per se. The polymer conjugate can include at least one recurring unit selected from Formulae (I), (II), (III) and (IV):

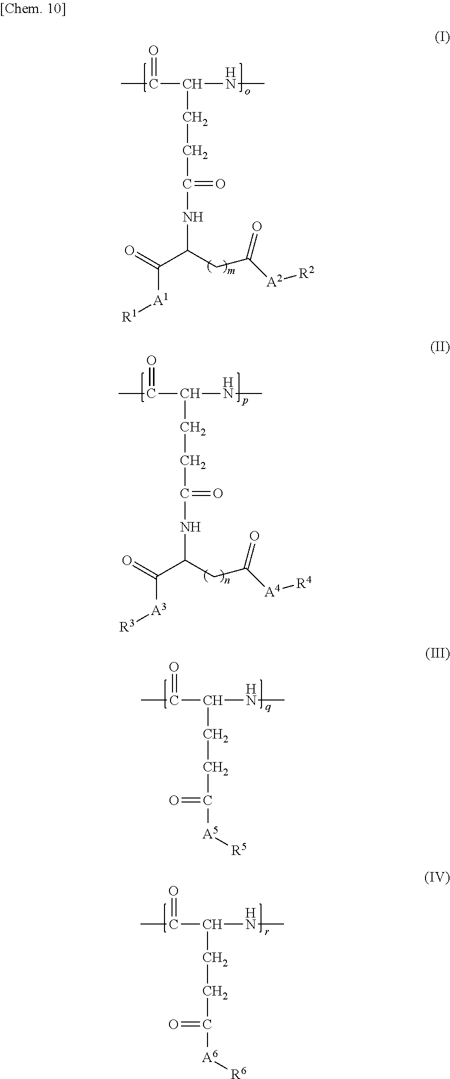

wherein: m can be independently 1 or 2; n can be independently 1 or 2; A¹ and A² can be each independently oxygen or NR⁷; A³ and A⁴ can be each independently oxygen or NR⁸; A⁵ and A⁶ can be each independently oxygen or NR⁹; R¹, R², R³, R⁴, R⁵ and R⁶ can be each independently selected from an optionally substituted C₁₋₁₀ alkyl, an optionally substituted C₆₋₂₀ aryl, ammonium, an alkali metal, a retinoid and a group that comprises a detectable label; R⁷, R⁸ and R⁹ can be each independently hydrogen or C₁₋₄ alkyl; o, p, q, and r can be each independently 0, 1 or greater, wherein the sum of o, p, q, and r is 2 or greater; and provided that at least one of R¹, R², R³, R⁴, R⁵ and R⁶ is a group that comprises a detectable label and at least one of R¹, R², R³, R⁴, R⁵ and R⁶ is a retinoid. Examples of alkali metal include lithium (Li), sodium (Na), potassium (K), rubidium (Rb), and cesium (Cs). In an embodiment, the alkali metal is sodium.

A broad variety of other recurring units may be included in the polymer conjugate comprising at least one recurring unit of selected from Formulae (I), (II), (III) and (IV). In some embodiments, a polymer conjugate described herein can further include a recurring unit of Formula (V):

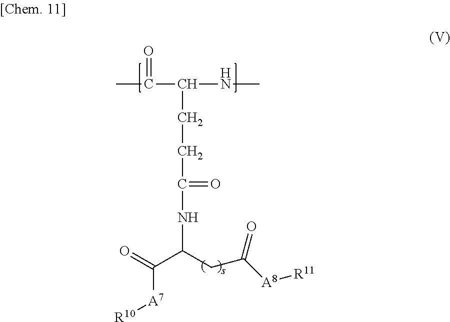

wherein: s can be independently 1 or 2; A⁷ and A⁸ can be each independently oxygen or NR¹²; R¹² can be hydrogen or C₁₋₄ alkyl; R¹⁰ and R¹¹ can be each independently selected from an optionally substituted C₁₋₁₀ alkyl, an optionally substituted C₆₋₂₀ aryl, ammonium and an alkali metal.

An embodiment provides a polymer conjugate as described herein which further can include a recurring unit of Formula (VI):

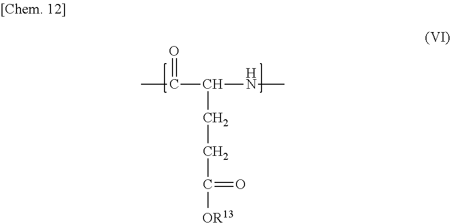

wherein R¹³ can be hydrogen, ammonium, or an alkali metal. When the R¹³ group is hydrogen, then the recurring unit of the Formula (VI) is a recurring unit of glutamic acid.

Various detectable labels can be a part of the polymer conjugates described herein, for example, a polymer conjugate containing at least one recurring unit selected from Formulae (I), (II), (III) and (IV). In some embodiments, the detectable label can include a metal. For example, the metal can be selected from Gd(III), Yttrium-88 and Indium-111. In some embodiments, the detectable label can include a ligand. Suitable ligands include, but are not limited to, diethylenetriaminepentacetic acid (DTPA), tetraazacyclododecane-1,4,7,10-tetraacetic acid (DOTA), (1,2-ethanediyldinitrilo)tetraacetate (EDTA), ethylenediamine, 2,2′-bipyridine (bipy), 1,10-phenanthroline (phen), 1,2-bis(diphenylphosphino)ethane (DPPE), 2,4-pentanedione (acac), and ethanedioate (ox). In an embodiment, the detectable label can include a ligand selected from diethylenetriaminepentacetic acid (DTPA) and tetraazacyclododecane-1,4,7,10-tetraacetic acid (DOTA).

A group that includes a paramagnetic metal chelate is one example of a suitable detectable label. In some embodiments, the paramagnetic metal chelate can include one of the following groups:

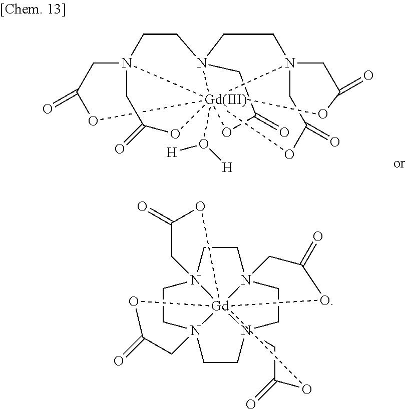

Another example of a suitable detectable label is a dye, such as Texas-Red or derivative thereof.

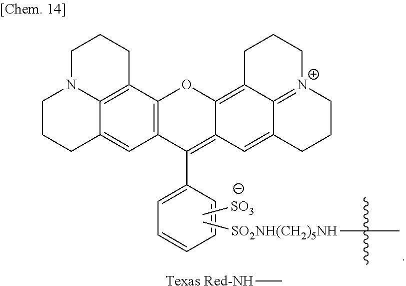

Various retinoids may be used in the polymer conjugates described herein, for example, a polymer conjugate containing at least one recurring unit of selected from Formulae (I), (II), (III) and (IV). Suitable retinoids include retinol, retinal, retinoic acid, rexinoid, or derivatives or analogs thereof. Exemplary retinols include vitamin A, all-trans retinol, retinyl palmitate, and retinyl acetate. One example of a retinal is 11-cis-retinal. Rexinoids are retinoid compounds which are selective for retinoid X receptors (RXR). An exemplary rexinoid is retinoid bexarotene. Other retinoid derivatives and analogs include etretinate, acitretin, tazarotene, bexarotene, adapalene, and fenretinide. In some embodiments, the retinoid can be selected from retinol, retinal, retinoic acid, all-trans retinol, all-trans retinoic acid, retinyl palmitate, 11-cis-retinal and 13-cis-retinoic acid. In an embodiment, the retinoid may include vitamin A. In another embodiment, the retinoid may include retinol.

The group that includes a detectable label may be conjugated to the polymer in many different ways. In an embodiment, the group that comprises a detectable label can be directly attached to the polymer. For example, the group that comprises a detectable label can be directly attached to a recurring unit of Formula (I), (II), (III) and/or (IV). In one embodiment, the group that comprises a detectable label can be directly attached to the polymer through an oxygen, a sulfur, a nitrogen and/or a carbon atom of the group that includes a detectable label. In other embodiments, the group that includes a detectable label can further include a linker group. In an embodiment, the group that includes a detectable label can be attached to the polymer, e.g., to a recurring unit of Formula (I), (II), (III) and/or (IV), through a linker group. The linker group may be relatively small. For instance, the linker group may comprise an amine, an amide, an ether, an ester, a hydroxyl group, a carbonyl group, or a thioether group. Alternatively, the linker group may be relatively large. For instance, the linker group may comprise an alkyl group, an aryl group, an aryl(C₁₋₆ alkyl) group (e.g., phenyl-(CH ₂)₁₋₄—), a heteroaryl group, or a heteroaryl(C₁₋₆ alkyl) group. In one embodiment, the linker can be —NH(CH₂)₁₋₄—NH—. In another embodiment, the linker can be —(CH₂)₁₋₄-aryl-NH—. As an example, the linker group can be attached in place of a hydrogen at a carbon of the group that includes a detectable label. The linker group can be added to the polymer, for example, to a recurring unit of Formula (I), (II), (III) and/or (IV), using methods known to those skilled in the art.

As with the group that includes a detectable label, the retinoid may be conjugated to the polymer in many different ways. In an embodiment, the retinoid can be directly attached to the polymer. For example, the retinoid can be directly attached to a recurring unit of Formula (I), (II), (III) and/or (IV). In one embodiment, the retinoid can be directly attached to the polymer through an oxygen, a sulfur, a nitrogen and/or a carbon atom of the retinoid. The retinoid can also be conjugated to the polymer, for example a polymer that includes at least one recurring of Formula (I), (II), (III) and/or (IV), through a linking group. The linking groups described herein with respect to the group that includes a detectable label can also be utilized with the retinoid. The linker group can be added to the polymer, for example, to a recurring unit of Formula (I), (II), (III) and/or (IV), and/or the retinoid using methods known to those skilled in the art.

In some embodiments, m in Formula (I) can be 1. In an embodiment, m in Formula (I) can be 2. In some embodiments, n in Formula (II) can be 1. In an embodiment, n in Formula (II) can be 2. In some embodiments, s in Formula (III) can be 1. In other embodiments, s in Formula (III) can be 2.

In some embodiments, the polymer conjugates described herein can include an alkali metal, for example, lithium (Li), sodium (Na), potassium (K), rubidium (Rb), and cesium (Cs). In an embodiment, the alkali metal may be sodium or potassium. In an embodiment, the alkali metal can be sodium.

Polymers that include at least two different recurring units of Formula (I), (II), (III) and/or (IV) are copolymers. Furthermore, polymers comprising at least one of recurring unit of the Formula (I), (II), (III) and (IV) may be copolymers that comprise other recurring units that are not of the Formula (I), (II), (III) and/or (IV).

The number of recurring units of Formula (I) and the number of recurring units of Formula (II) can be each independently selected, and may be varied over a broad range. In an embodiment, the number of recurring units of Formula (I) can be in the range of from about 50 to about 5,000, and more preferably from about 100 to about 2,000. Likewise, in some embodiments, the number of recurring units of Formula (II) can be in the range of from about 50 to about 5,000, and more preferably from about 100 to about 2,000.

Similarly, the number of recurring units of Formula (III) and the number of recurring units of Formula (IV) can be each be independently selected, and can vary. In an embodiment, the number of recurring units of Formula (III) can be in the range of from about 50 to about 5,000, and more preferably from about 100 to about 2,000. In some embodiments, the number of recurring units of Formula (IV) can be in the range of from about 50 to about 5,000, and more preferably from about 100 to about 2,000.

The percentage of recurring units of Formula (I) in the polymer conjugate, based on the total number of recurring units, may vary over a wide range. In an embodiment, the polymer conjugate may include up to about 99 mole % of the recurring unit of Formula (I), based on the total moles of recurring units in the polymer conjugate. In an embodiment, the polymer conjugate may include about 1 mole % to about 99 mole % of the recurring unit of Formula (I), based on the total moles of recurring units in the polymer conjugate. In an embodiment, the polymer conjugate may include about 1 mole % to about 50 mole % of the recurring unit of Formula (I) based on the total moles of recurring units of the polymer conjugate. In an embodiment, the polymer conjugate may include about 1 mole % to about 30 mole % of the recurring unit of Formula (I) based on the total moles of recurring units of the polymer conjugate. In an embodiment, the polymer conjugate may include about 1 mole % to about 20 mole % of the recurring unit of Formula (I) based on the total moles of recurring units of the polymer conjugate. In another embodiment, the polymer conjugate may include about 1 mole % to about 10 mole % of the recurring unit of Formula (I) based on the total moles of recurring units of the polymer conjugate.

The percentage of recurring units of Formula (II) in the polymer conjugate, based on the total number of recurring units, may also vary over a wide range. In an embodiment, the polymer conjugate may include up to about 99 mole % of the recurring unit of Formula (II), based on the total moles of recurring units in the polymer conjugate. In an embodiment, the polymer conjugate may include about 1 mole % to about 99 mole % of the recurring unit of Formula (II), based on the total moles of recurring units in the polymer conjugate. In an embodiment, the polymer conjugate may include about 1 mole % to about 50 mole % of the recurring unit of Formula (II) based on the total moles of recurring units of the polymer conjugate. In an embodiment, the polymer conjugate may include about 1 mole % to about 30 mole % of the recurring unit of Formula (II) based on the total moles of recurring units of the polymer conjugate. In an embodiment, the polymer conjugate may include about 1 mole % to about 20 mole % of the recurring unit of Formula (II) based on the total moles of recurring units of the polymer conjugate. In another embodiment, the polymer conjugate may include about 1 mole % to about 10 mole % of the recurring unit of Formula (II) based on the total moles of recurring units of the polymer conjugate.

The percentage of recurring units of Formula (III) in the polymer conjugate, based on the total number of recurring units, may vary over a wide range. In an embodiment, the polymer conjugate may include up to about 99 mole % of the recurring unit of Formula (III), based on the total moles of recurring units in the polymer conjugate. In an embodiment, the polymer conjugate may include about 1 mole % to about 99 mole % of the recurring unit of Formula (III), based on the total moles of recurring units in the polymer conjugate. In an embodiment, the polymer conjugate may include about 1 mole % to about 50 mole % of the recurring unit of Formula (III) based on the total moles of recurring units of the polymer conjugate. In an embodiment, the polymer conjugate may include about 1 mole % to about 30 mole % of the recurring unit of Formula (III) based on the total moles of recurring units of the polymer conjugate. In an embodiment, the polymer conjugate may include about 1 mole % to about 20 mole % of the recurring unit of Formula (III) based on the total moles of recurring units of the polymer conjugate. In another embodiment, the polymer conjugate may include about 1 mole % to about 10 mole % of the recurring unit of Formula (III) based on the total moles of recurring units of the polymer conjugate.

Likewise, the percentage of recurring units of Formula (IV) in the polymer conjugate, based on the total number of recurring units, may vary over a wide range. In an embodiment, the polymer conjugate may include up to about 99 mole % of the recurring unit of Formula (IV), based on the total moles of recurring units in the polymer conjugate. In an embodiment, the polymer conjugate may include about 1 mole % to about 99 mole % of the recurring unit of Formula (IV), based on the total moles of recurring units in the polymer conjugate. In an embodiment, the polymer conjugate may include about 1 mole % to about 50 mole % of the recurring unit of Formula (IV) based on the total moles of recurring units of the polymer conjugate. In an embodiment, the polymer conjugate may include about 1 mole % to about 30 mole % of the recurring unit of Formula (IV) based on the total moles of recurring units of the polymer conjugate. In an embodiment, the polymer conjugate may include about 1 mole % to about 20 mole % of the recurring unit of Formula (IV) based on the total moles of recurring units of the polymer conjugate. In another embodiment, the polymer conjugate may include about 1 mole % to about 10 mole % of the recurring unit of Formula (IV) based on the total moles of recurring units of the polymer conjugate.

If one or more recurring units of Formula (V) are present in the polymer conjugate, the percentage of recurring units of Formula (V) in the polymer conjugate, based on the total number of recurring units, may vary over a wide range. Similarly, if one or more recurring units of Formula (VI) are present in the polymer conjugate, the percentage of recurring units of Formula (VI) in the polymer conjugate, based on the total number of recurring units, may vary over a wide range. Exemplary embodiments are shown in Table 1.

TABLE 1

| Recurring Unit | Mole Percent in the Polymer Conjugate |
|---|---|
| Formula (V) | about 1 mole % to about 99 mole % |
| | about 1 mole % to about 50 mole % |
| | about 1 mole % to about 30 mole % |
| | about 1 mole % to about 20 mole % |
| | about 1 mole % to about 10 mole % |
| Formula (VI) | about 1 mole % to about 99 mole % |
| | about 1 mole % to about 50 mole % |
| | about 1 mole % to about 30 mole % |
| | about 1 mole % to about 20 mole % |
| | about 1 mole % to about 10 mole % |

The mole percentages are based on the total moles of recurring units in the polymer conjugate.

In an embodiment, a detectable label can be non-covalently encapsulated or partially encapsulated within a polymer matrix of a polymer conjugate described herein. For example, polymer conjugates described herein may be present in various forms, including in the form of particles, flakes, rods, fibers, films, foams, suspensions (in liquid or gas), a gel, a solid, or a liquid. The size and shape of these various forms is not limited. Free and non-conjugated detectable label(s), such as those described herein, may be mixed with a polymer conjugate described herein as it forms a matrix and be non-covalently encapsulated or partially encapsulated therein. Similarly, the retinoid can be non-covalently encapsulated or partially encapsulated within a polymer matrix of the polymer conjugate described herein.

The amount of detectable label in the polymer conjugate may vary over a wide range. In an embodiment, the polymer conjugate can include a total amount of the detectable label in the range of about 0.5% to about 50% (weight/weight) based on the mass ratio of the detectable label to the polymer conjugate (the weight of the detectable label is accounted for in the polymer conjugate). In an embodiment, the polymer conjugate can include an amount of the detectable label in the range of about 1% to about 40% (weight/weight) based on the mass ratio of the detectable label to the polymer conjugate (same basis). In an embodiment, the polymer conjugate can include an amount of the detectable label in the range of about 1% to about 30% (weight/weight) based on the mass ratio of the detectable label to the polymer conjugate (same basis). In an embodiment, the polymer conjugate can include an amount of the detectable label in the range of about 1% to about 20% (weight/weight) based on the mass ratio of the detectable label to the polymer conjugate (same basis). In an embodiment, the polymer conjugate can include an amount of the detectable label in the range of about 1% to about 10% (weight/weight) based on the mass ratio of the detectable label to the polymer conjugate (basis).

The amount of retinoid present in the polymer conjugate can also vary over a wide range. In some embodiments, the retinoid can be about 1% to about 50% (weight/weight) of the total mass of the polymer conjugate (wherein the mass of the retinoid is included in the total mass of the polymer conjugate). In other embodiments, the retinoid may be about 10% to about 30% w/w of the total mass of the polymer conjugate (same basis). In still other embodiments, the retinoid may be about 20% to about 40% w/w of the total mass of the polymer conjugate (same basis).

The amount of the detectable label, the amount of the retinoid, and the percentage amounts of the recurring units of the Formula (I), (II), (III) and/or (IV) can be preferably selected to provide a polymer conjugate solubility that is greater than that of a comparable polyglutamic acid conjugate that comprises substantially the same amount of the same agent(s). In an embodiment, the polymer conjugate solubility is greater than that of a comparable polyglutamic acid conjugate. Solubility is measured by forming a polymer conjugate solution comprising at least 5 mg/mL of the polymer conjugate in 0.9 wt. % aqueous NaCl at about 22 degree Celsius (° C.), and determining the optical clarity. Optical clarity may be determined turbidimetrically, e.g., by visual observation or by appropriate instrumental methods known to those skilled in the art. Comparison of the resulting solubility to a similarly formed polyglutamic acid conjugate solution shows improved solubility as evidenced by greater optical clarity over a broader range of pH values. Thus, a polymer conjugate solubility is greater than that of a comparable polyglutamic acid conjugate that comprises substantially the same amount of the agent when a tested polymer conjugate solution, comprising at least 5 mg/mL of the polymer conjugate in 0.9 wt. % aqueous NaCl at about 22° C., has greater optical clarity over a broader pH range than that of a comparable tested polyglutamic acid conjugate solution. Those skilled in the art will understand that a “comparable” polyglutamic acid conjugate is a control material in which the polymeric portion of the conjugate has a molecular weight that is approximately the same as that of the subject polymer conjugate (comprising a recurring unit of the Formula (I), (II), (III) and/or (IV)) to which it is being compared.

The polymer conjugate can contain one or more chiral carbon atoms. The chiral carbon (which may be indicated by an asterisk *) can have the rectus (right handed) or the sinister (left handed) configuration, and thus the recurring unit may be racemic, enantiomeric or enantiomerically enriched. The symbols “n” and “*” (designating a chiral carbon), as used elsewhere herein, have the same meaning as specified above, unless otherwise stated.

Polymer conjugates comprising at least one recurring unit selected from Formula (I), (II), (III) and (IV) may be prepared in various ways. In an embodiment, a first polymeric reactant can be dissolved or partially dissolved in a solvent to form a first dissolved or partially dissolved polymeric reactant. The first dissolved or partially dissolved polymeric reactant can be then reacted with a second reactant to form a second polymeric reactant. In an embodiment, the second reactant can include a group that comprises a detectable label. In another embodiment, the second reactant can include a retinoid. In an embodiment, the retinoid can be retinol or a derivative thereof. In yet still another embodiment, the second reactant can include a ligand such as diethylenetriaminepentacetic acid (DTPA), tetraazacyclododecane-1,4,7,10-tetraacetic acid (DOTA), (1,2-ethanediyldinitrilo)tetraacetate (EDTA), ethylenediamine, 2,2′-bipyridine (bipy), 1,10-phenanthroline (phen), 1,2-bis(diphenylphosphino)ethane (DPPE), 2,4-pentanedione (acac), and ethanedioate (ox). In an embodiment, the second reactant can include a substituent selected from a hydroxy and an amine.

The first polymeric reactant may include any suitable material capable of forming a polymer comprising at least one recurring unit selected from Formula (I), (II), (III) and (IV). In an embodiment, the polymeric reactant may include a recurring unit of Formula (VII):

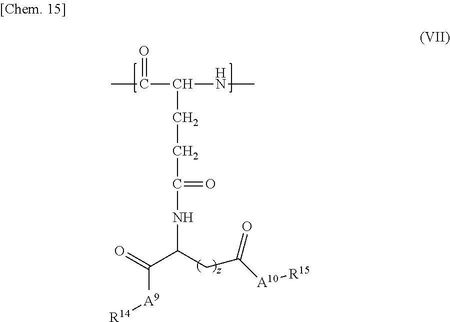

wherein z can be independently 1 or 2; A⁹ and A¹⁰ can be each oxygen; and R¹⁴ and R¹⁵ can be each independently selected from hydrogen, ammonium, and an alkali metal.

In another embodiment, the first polymeric reactant may include a recurring unit of Formula (VIII):

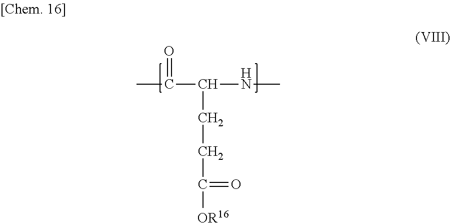

wherein R¹⁶ can be selected from hydrogen, ammonium, and an alkali metal.

The second polymeric reactant can be then reacted with a third reactant to form an intermediate product or, in some embodiments, a polymer comprising at least one recurring unit selected from Formula (I), (II), (III) and (IV). If necessary or desired, the second polymeric reactant can be dissolved or partially dissolved in a solvent to form a second dissolved or partially dissolved polymeric reactant. In an embodiment, the third reactant can include a group that comprises a detectable label. In another embodiment, the third reactant can include a retinoid. In an embodiment, the retinoid can be retinol or a derivative thereof. In yet still other embodiments, the third reactant can include a ligand such as those described with respect to the second reactant. In an embodiment, the second reactant can include a substituent selected from a hydroxy and an amine.

If the second or third reactant is a ligand, a fourth reactant can be added after the addition of the ligand in which the fourth reactant includes a metal. Exemplary metals include, but are not limited to, Gd(III), Yttrium-88 and Indium-111.

Mixtures of free detectable labels and/or retinoids with the polymer conjugates described herein may be formed in various ways, e.g., to form a matrix in which some or all of the detectable labels and/or retinoids are non-covalently encapsulated or partially encapsulated therein. Such a mixture may contain, for example, both conjugated and non-conjugated detectable label(s) and/or retinoid(s).

The polymer reactants may be dissolved or partially dissolved in a variety of solvents to prepare it for mixing with the group that comprises a detectable label, the retinoid and/or the ligand. In an embodiment, the solvent can include a hydrophilic solvent, such as a polar solvent. Suitable polar solvents include protic solvents such as water, methanol, ethanol, propanol, isopropanol, butanol, formic acid, and acetic acid. Other suitable polar solvents include aprotic solvents, such as acetone, acetonitrile, dimethylformamide, dimethyl sulfoxide, tetrahydrofuran, and 1,4-dioxane. In an embodiment, the solvent can be an aqueous solvent, for example, water.

Dissolving or partial dissolving the polymer reactants in a solvent may be further aided by the use of conventional mechanical techniques. For instance, the polymer conjugate may be shaken or stirred in the solvent to induce dissolving or partial dissolving. In an embodiment, the polymer and solvent are sonicated. Sonication is the act of applying sound energy, for example, ultrasound energy, to agitate the particles in a sample. Sonication may take place using, for example, an ultrasonic bath or an ultrasonic probe. The degree to which the polymer is dissolved may be controlled by varying the intensity and duration of the mechanical shaking or stirring or the sonication conditions. Shaking, stirring, or sonicating may take place over any duration of time. For example, the mixture may be sonicated for a period of time ranging between several seconds to several hours. In an embodiment, the polymer conjugate can be sonicated in the solvent for a period of time ranging between about 1 minute and about 10 minutes. In an embodiment, the polymer conjugate can be sonicated in the solvent for about 5 minutes.

In an embodiment, the group that comprises a detectable label and/or the ligand can be added to the polymer conjugate solution. The group that comprises a detectable label and/or the ligand may or may not be dissolved or partially dissolved in solvent(s) before it is mixed with the polymer conjugate. If the group that comprises a detectable label and/or the ligand is dissolved or partially dissolved in a solvent, the solvent may include a hydrophilic solvent, such as a polar solvent. Suitable polar solvents include protic solvents such as water, methanol, ethanol, propanol, isopropanol, butanol, formic acid, acetic acid, and acetone. Other suitable polar solvents include aprotic solvents, such as acetone, acetonitrile, dimethylformamide, dimethyl sulfoxide, tetrahydrofuran, and 1,4-dioxane.

Likewise, in an embodiment, the retinoid can be added to the polymer reactant solution. The retinoid may or may not be dissolved or partially dissolved in solvent(s) before it is mixed with the polymer reactant. If the retinoid is dissolved or partially dissolved in a solvent, the solvent may include a hydrophilic solvent, such as a polar solvent. Suitable polar solvents include protic solvents such as water, methanol, ethanol, propanol, isopropanol, butanol, formic acid, acetic acid, and acetone. Other suitable polar solvents include aprotic solvents, such as acetone, acetonitrile, dimethylformamide, dimethyl sulfoxide, tetrahydrofuran, and 1,4-dioxane.

After the group that comprises the group that comprises a detectable label, the retinoid and/or the ligand is added to the polymer reactant solution, for example, by using a pipette, additional mixing may be performed. For instance, the solution comprising the polymer reactant and the group that comprises a detectable label may be shaken or stirred. In an embodiment, the solution comprising the polymer conjugate and the group that comprises a detectable label can be sonicated. Shaking, stirring, or sonicating may take place over any duration of time. For instance, the mixture may be sonicated for a period of time ranging between several seconds to several hours.

In an embodiment, the polymer reactant and the group that comprises a detectable label, the retinoid and/or the ligand are mixed together before either is dissolved in a solvent. In an embodiment, a solvent or mixture of solvents may be added to the mixture of the polymer reactant and the group that comprises a detectable label, the retinoid and/or the ligand. After the solvent or mixture of solvents is added to the polymer reactant and the group that comprises a detectable label, the retinoid and/or the ligand, one or more of the polymer reactant and the group that comprises a detectable label, the retinoid and/or the ligand may be dissolved or partially dissolved. The solvent or mixture of solvents may include one or more of water, methanol, ethanol, propanol, isopropanol, butanol, formic acid, acetic acid, acetone, acetonitrile, dimethylformamide, dimethyl sulfoxide, tetrahydrofuran, and 1,4-dioxane. In an embodiment, the mixture of solvents can include an alcohol and water. In an embodiment, the mixture of solvents can include ethanol and water.

Optionally, the polymer conjugate comprising the group that comprises a detectable label and the retinoid may then be isolated and/or purified. Suitable methods known to those skilled in the art can be used to isolate and/or purify the polymer conjugates described herein. The polymer conjugates may then be dried by any suitable method known to those skilled in the art. For example, in one embodiment, the polymer conjugate can be freeze-dried. The conditions of freeze-drying the composition may vary. In an embodiment, the mixture can be freeze-dried at a temperature ranging between about −30° C. to about −10° C. In an embodiment, the mixture can be freeze-dried at a temperature of about −20° C. Once the polymer conjugate comprising the group that comprises a detectable label and the retinoid has been optionally isolated and dried, it may then be stored in appropriate conditions. For example, the composition may be stored at a temperature suitable for freeze-drying, as set forth above.

The reaction with a third reactant may take place before, at about the same time, or after the dissolved or partially dissolved polymeric reactant is reacted with the second reactant. In some embodiments, the dissolved or partially dissolved polymer reactant can be reacted with at least a portion of the second reactant before reacting with the third reactant. In an embodiment, the intermediate compound that forms after the addition of at least a portion of the second reactant can be isolated before adding the third reactant. In another embodiment, the third reactant can be added without isolating the intermediate compound that forms after the addition of the second reactant. In other embodiments, the dissolved or partially dissolved polymer reactant can be reacted with at least a portion of the second reactant at about the same time as reacting with the third reactant. In an embodiment, the dissolved or partially dissolved polymer reactant can be reacted with at least a portion of the second reactant after reacting with the third reactant. In an embodiment, the intermediate compound that forms after the addition of at least a portion of the third reactant can be isolated before adding the second reactant.

If a fourth reactant that includes a metal is added, in some embodiments, the fourth reactant can be added at about the same time as the third and/or second reactant. In an embodiment, the fourth reactant can be added after as the third and/or second reactant. In an embodiment, the intermediate compound with a ligand such as those described herein can be isolated before adding the fourth reactant. In another embodiment, the fourth reactant can be added to the intermediate compound with a ligand without isolating the intermediate compound.

In an embodiment, a method of making the polymer conjugate can include reacting the dissolved or partially dissolved polymeric reactant with the second reactant and/or third reactant in the presence of a coupling agent. Any suitable coupling agent may be used. In an embodiment, the coupling agent is selected from 1-ethyl-3-(3-dimethylaminopropyl)-carbodiimide (EDC), 1,3-dicyclohexyl car-bodiimide (DCC), 1,1′-carbonyl-diimidazole (CDI), N,N′-disuccinimidyl carbonate (DSC), N-[(dimethylamino)-1H-1,2,3-triazolo-[4,5-b]pyridine-1-yl-methylene]-N-methylmethan aminium hexafluorophosphate N-oxide (HATU), 2-[(1H-benzotriazol-1-yl)-1,1,3,3-tetramethylaminium hexafluorophosphate (HBTU), 2-[(6-chloro-1H-benzotriazol-1-yl)-1,1,3,3-tetramethylaminium hexafluorophosphate (HCTU), benzotriazole-1-yl-oxy-tris-pyrrolidino-phosphonium hexafluorophosphate (PyBOP®), bromo-tris-pyrrolidino-phosphonium hexafluorophosphate (PyBroP®), 2-[(1H-benzotriazol-1-yl)-1,1,3,3-tetramethylaminium tetrafluoroborate (TBTU), and benzotriazol-1-yl-oxy-tris-(dimethylamino)phosphonium hexafluorophosphate (BOP).

Any suitable solvent that allows the reaction to take place may be used. In an embodiment, the solvent may be a polar aprotic solvent. For instance, the solvent may be selected from N,N-dimethylformamide (DMF), dimethyl sulfoxide (DMSO), N-methyl-2-pyridone (NMP), and N,N-dimethylacetamide (DMAc).

In another embodiment, the reaction may further include reacting the dissolved or partially dissolved polymeric reactant in the presence of a catalyst. Any catalyst that promotes the reaction may be used. In an embodiment, the catalyst may comprise 4-dimethylaminopyridine (DMAP).

In an embodiment, a polymer comprising at least one recurring unit selected from Formula (I) and Formula (II) can be produced starting with polyglutamic acid and an amino acid such as asparatic and/or glutamic acid. Alternatively, in another embodiment, the polymer may be created by first converting the starting polyglutamic acid material into its salt form. The salt form of polyglutamic acid can be obtained by reacting polyglutamic acid with a suitable base, e.g., sodium bicarbonate. An amino acid moiety can be attached to the pendant carboxylic acid group of the polyglutamic acid. The weight average molecular weight of the polyglutamic acid may vary over a broad range, but is preferably from about 10,000 to about 500,000 daltons, and more preferably from about 25,000 to about 300,000 daltons. Such a reaction may be used to create poly-(gamma-L-aspartyl-glutamine) or poly-(gamma-L-glutamyl-glutamine).

In an embodiment, the amino acid is protected by a protecting group before attachment to the polyglutamic acid. One example of a protected amino acid moiety suitable for this reaction is L-aspartic acid di-t-butyl ester hydrochloride, shown below:

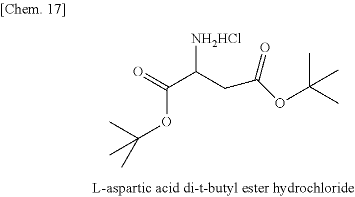

Reaction of the polyglutamic acid with the amino acid may take place in the presence of any suitable solvent. In an embodiment, the solvent can be an aprotic solvent. In a preferred embodiment, the solvent can be N,N′-dimethylformamide.

In an embodiment, a coupling agent such as EDC, DCC, CDI, DSC, HATU, HBTU, HCTU, PyBOP®, PyBroP®, TBTU, and BOP can be used. In other embodiments, polyglutamic acid and an amino acid can be reacted using a catalyst (e.g., DMAP).

After completion of the reaction, if the oxygen atoms of the amino acid are protected, the protecting groups can be removed using known methods such as using a suitable acid (e.g., trifluoroacetic acid). If desired, the salt form of the polymer obtained from reacting polyglutamic acid with the amino acid can be formed by treating the acid form of the polymer with a suitable base solution, e.g., sodium bicarbonate solution.

The polymer may be recovered and/or purified by methods known to those skilled in the art. For example, the solvent may be removed by suitable methods, for instance, rotary evaporation. Additionally, the reaction mixture may be filtered into an acidic water solution to induce precipitation. The resultant precipitate can then be filtered, and washed with water.

In some embodiments, a polymer comprising at least one recurring unit selected from Formula (I) and Formula (II) can also include a recurring unit of Formula (VI) as set forth above. One method for forming such a polymer is by starting with polyglutamic acid and reacting it with an amino acid such as asparatic and/or glutamic acid, in an amount that is less than 1.0 equivalents of the amino acid based on polyglutamic acid. For example, in one embodiment, 0.7 equivalents of an amino acid based on the polyglutamic acid can be reacted with polyglutamic acid, so that about 70% of the recurring units of the resulting polymer comprise the amino acid. As discussed above, the oxygen atoms of the amino acid can be protected using a suitable protecting group. In an embodiment, the amino acid may be L-aspartic acid or L-glutamic acid. In another embodiment, the oxygen atoms of the amino acid can be protected with t-butyl groups. If the oxygen atoms of the amino acid are protected, the protecting groups can be removed using known methods such as a suitable acid (e.g., trifluoroacetic acid).

In some embodiments, a polymer comprising at least one recurring unit selected from Formula (III) and Formula (IV) can be produced starting with polyglutamic acid. As described previously, a polymer comprising at least one recurring unit selected from Formula (III) and (IV) can also include a recurring unit of Formula (V). A method for forming such a polymer is by starting with polyglutamic acid and/or its salt and adding less then 1.0 equivalents of an amino acid such as L-aspartic acid or L-glutamic acid.

Polymers comprising recurring units of one or more recurring units selected from Formula (I), (II), (III), (IV), (V) and (VI) can be synthesized by using various corresponding starting monomers using methods known to those skilled in the art.

Conjugation of the group that comprises a detectable label to the polymer acid or its salt form may be carried out in various ways, e.g., by covalently bonding the group comprising a detectable label to various polymers. Similarly, a retinoid can be conjugated to the polymer acid or its salt in various ways. One method for conjugating the aforementioned groups to the polymer is by using heat (e.g., heat from using a microwave method). Alternatively, conjugation may take place at room temperature. Appropriate solvents, coupling agents, catalysts, and/or buffers as generally known to those skilled in the art and/or as described herein may be used to form the polymer conjugate.

In one embodiment, a polymer described herein (e.g., a polymer comprising a recurring unit selected from Formula (I), (II), (III) and/or (IV)) can be conjugated to a detectable label such as those described herein. In an embodiment, the detectable label can be Texas Red-NH₂.

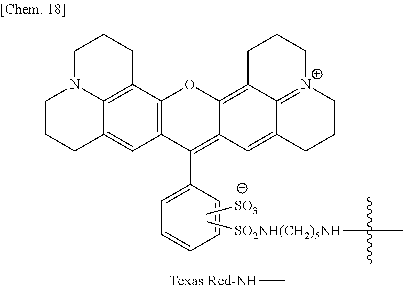

In one particular embodiment, a polymer comprising at least one recurring unit selected from Formula (I), Formula (II), and Formula (V) may be reacted with DCC, Texas Red-NH₂ dye, pyridine, and 4-dimethylaminopyridine. The mixture can be heated using a microwave method. In an embodiment, the reaction can be heated up to a temperature in the range of about 100° C.-150° C. In another embodiment, the time the materials can be heated ranges from 5 to 40 minutes. If desired, the reaction mixture can be cooled to room temperature. Suitable methods known to those skilled in the art can be used to isolate and/or purify the polymer conjugate. For instance, reaction mixture can be filtered into an acidic water solution. Any precipitate that forms can then be filtered and washed with water. Optionally, the precipitate can be purified by any suitable method. For example, the precipitate can be transferred into acetone and dissolved, and the resulting solution can be filtered again into a sodium bicarbonate solution. If desired, the resulting reaction solution can be dialyzed in water using a cellulose membrane and the polymer can be lyophilized and isolated.

Alternatively, the group that comprises a detectable label and/or the retinoid can be reacted with an amino acid such as glutamic and/or aspartic acid in which the group that comprises a detectable label and/or retinoid is coupled (e.g., covalently bonded) to the amino acid. The amino acid-label and/or amino-acid-retinoid compound can then be reacted with polyglutamic acid or its salt to form a polymer conjugate that includes at least one recurring unit selected from Formula (II) and Formula (II). The group that comprises a detectable label and/or retinoid can also be attached to a monomer that will be used to form part of the polymer conjugate such as a polymer conjugate that includes a recurring unit selected from Formula (I), (II), (III) and (IV). The monomer can then be polymerized using methods known to those skilled in the art to form the polymer conjugate. For example, a group that comprises a detectable label and/or a retinoid can be attached to glutamic acid prior to polymerization. Similarly, a group that comprises a detectable label and/or a retinoid can be attached to L-gamma-glutamylglutamine and/or gamma-L-aspartylglutamine. The resulting monomer with the attached group that comprises a detectable label and/or a retinoid can then be polymerized using methods known to those skilled in the art to form the polymer conjugate.

After formation of the polymer conjugate, any free amount of agent not covalently bonded to the polymer may also be measured. Methods known to those skilled in the art may be used to confirm the substantial absence of free detectable label and/or retinoid.

The polymer conjugates described above may be formed into nanoparticles in aqueous solution. Polymer conjugates (e.g., a polymer conjugate that includes at least one recurring unit selected from Formula (I), (II), (III) and (IV)) may be formed into nanoparticles in a similar manner. Such nanoparticles may be used to preferentially deliver a detectable label to a selected tissue.

The imaging agent and the compound (e.g., a polymer conjugate that includes at least one recurring unit selected from Formulae (I), (II), (III) and (IV)) of the present invention may be used to diagnose fibrotic disease. Thus, the present invention further relates to a diagnostic agent of fibrotic disease comprising the imaging agent and/or the compound or the invention, as well as to a method for diagnosing fibrotic disease comprising a step of administering an effective amount of the imaging agent, the compound or said diagnostic agent of the invention to a subject in need thereof, and a step of detecting the label contained in the administered imaging agent, compound or diagnostic agent.

An embodiment provides a composition that can include the agent (e.g., the imaging agent or the diagnostic agent) and/or the compound (e.g., a polymer conjugate that includes at least one recurring unit selected from Formulae (I), (II), (III) and (IV)) described herein and at least one selected from a pharmaceutically acceptable excipient, a carrier, and a diluent. In some embodiments, prodrugs, metabolites, stereoisomers, hydrates, solvates, polymorphs, and pharmaceutically acceptable salts of the compounds disclosed herein (e.g., a polymer conjugate that includes at least one recurring unit selected from Formula (I), (II), (III) and (IV)) are provided.

A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. An example, without limitation, of a prodrug would be a compound which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety. Conventional procedures for the selection and preparation of suitable prodrug derivatives are described, for example, in Design of Prodrugs, (ed. H. Bundgaard, Elsevier, 1985), which is hereby incorporated herein by reference in its entirety.

The term “pro-drug ester” refers to derivatives of the compounds disclosed herein formed by the addition of any of several ester-forming groups that are hydrolyzed under physiological conditions. Examples of pro-drug ester groups include pivaloyloxymethyl, acetoxymethyl, phthalidyl, indanyl and methoxymethyl, as well as other such groups known in the art, including a (5-R-2-oxo-1,3-dioxolen-4-yl)methyl group. Other examples of pro-drug ester groups can be found in, for example, T. Higuchi and V. Stella, in “Pro-drugs as Novel Delivery Systems”, Vol. 14, A.C.S. Symposium Series, American Chemical Society (1975); and “Bioreversible Carriers in Drug Design: Theory and Application”, edited by E. B. Roche, Pergamon Press: New York, 14-21 (1987) (providing examples of esters useful as prodrugs for compounds containing carboxyl groups). Each of the above-mentioned references is herein incorporated by reference in their entirety.

The term “pharmaceutically acceptable salt” refers to a salt of a compound that does not cause significant irritation to an organism to which it is administered and does not abrogate the biological activity and properties of the compound. In some embodiments, the salt is an acid addition salt of the compound. Pharmaceutical salts can be obtained by reacting a compound with inorganic acids such as hydrohalic acid (e.g., hydrochloric acid or hydrobromic acid), sulfuric acid, nitric acid, phosphoric acid and the like. Pharmaceutical salts can also be obtained by reacting a compound with an organic acid such as aliphatic or aromatic carboxylic or sulfonic acids, for example acetic, succinic, lactic, malic, tartaric, citric, ascorbic, nicotinic, methanesulfonic, ethane-sulfonic, p-toluensulfonic, salicylic or naphthalenesulfonic acid. Pharmaceutical salts can also be obtained by reacting a compound with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, C₁-C₇ alkylamine, cyclohexylamine, triethanolamine, ethylenediamine, and salts with amino acids such as arginine, lysine, and the like.

If the manufacture of pharmaceutical formulations involves intimate mixing of the pharmaceutical excipients and the active ingredient in its salt form, then it may be desirable to use pharmaceutical excipients which are non-basic, that is, either acidic or neutral excipients.

In various embodiments, the agents or the compounds disclosed herein (e.g., a polymer conjugate that includes at least one recurring unit selected from Formula (I), (II), (III) and (IV)) can be used alone, in combination with other agents or compounds disclosed herein, or in combination with one or more other agents active in the therapeutic areas described herein.

In another aspect, the present disclosure relates to a pharmaceutical composition comprising one or more physiologically acceptable surface active agents, carriers, diluents, excipients, smoothing agents, suspension agents, film forming substances, and coating assistants, or a combination thereof; and an agent and/or a compound (e.g., a polymer conjugate that includes at least one recurring unit selected from Formula (I), (II), (III) and (IV)) disclosed herein. Acceptable carriers or diluents for therapeutic use are well known in the pharmaceutical art, and are described, for example, in Remington's Pharmaceutical Sciences, 18th Ed., Mack Publishing Co., Easton, Pa. (1990), which is incorporated herein by reference in its entirety. Preservatives, stabilizers, dyes, sweeteners, fragrances, flavoring agents, and the like may be provided in the pharmaceutical composition. For example, sodium benzoate, ascorbic acid and esters of p-hydroxybenzoic acid may be added as preservatives. In addition, antioxidants and suspending agents may be used. In various embodiments, alcohols, esters, sulfated aliphatic alcohols, and the like may be used as surface active agents; sucrose, glucose, lactose, starch, crystallized cellulose, mannitol, light anhydrous silicate, magnesium aluminate, magnesium metasilicate aluminate, synthetic aluminum silicate, calcium carbonate, sodium acid carbonate, calcium hydrogen phosphate, calcium carboxymethyl cellulose, and the like may be used as excipients; magnesium stearate, talc, hardened oil and the like may be used as smoothing agents; coconut oil, olive oil, sesame oil, peanut oil, soya may be used as suspension agents or lubricants; cellulose acetate phthalate as a derivative of a carbohydrate such as cellulose or sugar, or methylacetate-methacrylate copolymer as a derivative of polyvinyl may be used as suspension agents; and plasticizers such as ester phthalates and the like may be used as suspension agents.

The term “pharmaceutical composition” refers to a mixture of an agent and/or a compound disclosed herein (e.g., a polymer conjugate that includes at least one recurring unit selected from Formula (I), (II), (III) and (IV)) with other chemical components, such as diluents or carriers. The pharmaceutical composition facilitates administration of the agent and/or the compound to an organism. Multiple techniques of administering an agent and/or a compound exist in the art including, but not limited to, oral, injection, aerosol, parenteral, and topical administration. Pharmaceutical compositions can also be obtained by reacting agents and/or compounds with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid and the like.

The term “carrier” used with respect to pharmaceutical composition refers to a chemical compound that facilitates the incorporation of an agent and/or a compound into cells or tissues. For example dimethyl sulfoxide (DMSO) is a commonly utilized carrier as it facilitates the uptake of many organic compounds into the cells or tissues of an organism.

The term “diluent” refers to chemical compounds diluted in water that will dissolve the agent and/or the compound of interest (e.g., a polymer conjugate that includes at least one recurring unit selected from Formula (I), (II), (III) and (IV)) as well as stabilize the biologically active form of the agent and/or the compound. Salts dissolved in buffered solutions are utilized as diluents in the art. One commonly used buffered solution is phosphate buffered saline because it mimics the salt conditions of human blood. Since buffer salts can control the pH of a solution at low concentrations, a buffered diluent rarely modifies the biological activity of an agent and/or a compound. The term “physiologically acceptable” refers to a carrier or diluent that does not abrogate the biological activity and properties of the agent and/or the compound.

In the agent of the present invention, the label may be contained within the interior of the agent, may be attached to the exterior thereof, or may be mixed therewith, as long as retinoid is present in such a configuration that it can function as targeting molecule. Accordingly, the present agent may be covered with an appropriate material such as, for example, an enteric coating or a material that disintegrates over time, or may be incorporated into an appropriate drug release system.

The pharmaceutical compositions described herein can be administered to a human patient per se, or in pharmaceutical compositions where they are mixed with other active ingredients, as in combination therapy, or suitable carriers or excipient(s). Techniques for formulation and administration of the agent and/or the compounds of the instant application may be found, e.g., in “Remington's Pharmaceutical Sciences,” Mack Publishing Co., Easton, Pa., 18th edition, 1990, and Hyojun Yakuzaigaku (Standard Pharmaceutics), Ed. by Yoshiteru Watanabe et al., Nankodo, 2003.

Suitable routes of administration may, for example, include oral, rectal, transmucosal, transdermal, transnasal, intraauricular, topical, or intestinal administration; parenteral delivery, including intramuscular, subcutaneous, intravenous, intraarterial, intraportal, intra-lymphatic, intra-lymph node, intramedullary, intrathecal, direct intraventricular, intracerebroventricular, intraperitoneal, intranasal, intracerebral, intraocular injections, as well as intrapulmonary, intra-airway, intratracheal, intra-bronchial, intrauterine, or intratracheal administration. In some embodiments, the agent and/or the compounds (e.g., a polymer conjugate that includes at least one recurring unit selected from Formula (I), (II), (III) and (IV)) can also be administered in sustained or controlled release dosage forms, including depot injections, osmotic pumps, pills, transdermal (including electrotransport) patches, and the like, for prolonged and/or timed, pulsed administration at a predetermined rate.

The pharmaceutical compositions may be formulated into a dosage form suitable for each administration route. Such a dosage form and formulation method may be selected as appropriate from any known forms and methods.

Examples of dosage forms suitable for oral administration include, but are not limited to, powder, granules, tablet, capsule, liquid, suspension, emulsion, gel, and syrup, and examples of the dosage form suitable for parenteral administration include injections such as an injectable solution, an injectable suspension, an injectable emulsion, and an injection in a form that is prepared at the time of use. Formulations for parenteral administration may be a configuration such as an aqueous or nonaqueous isotonic aseptic solution or suspension.

The pharmaceutical compositions may be manufactured in a manner that is itself known, e.g., by means of conventional mixing, dissolving, granulating, dragee-making, levigating, emulsifying, encapsulating, entrapping or tabletting processes.

Pharmaceutical compositions may be formulated in a conventional manner using one or more physiologically acceptable carriers comprising excipients and auxiliaries which facilitate processing of the active compounds into preparations which can be used pharmaceutically. Proper formulation is dependent upon the route of administration chosen. Any of the well-known techniques, carriers, and excipients may be used as suitable and as understood in the art; e.g., in Remington's Pharmaceutical Sciences, and in Standard Pharmaceutics above.

Injectables can be prepared in conventional forms, either as liquid solutions or suspensions, solid forms suitable for solution or suspension in liquid prior to injection, or as emulsions. Suitable excipients are, for example, water, saline, dextrose, mannitol, lactose, lecithin, albumin, sodium glutamate, cysteine hydrochloride, and the like. In addition, if desired, the injectable pharmaceutical compositions may contain minor amounts of nontoxic auxiliary substances, such as wetting agents, pH buffering agents, and the like. Physiologically compatible buffers include, but are not limited to, Hanks's solution, Ringer's solution, or physiological saline buffer. If desired, absorption enhancing preparations (for example, liposomes), may be utilized.

For transmucosal administration, penetrants appropriate to the barrier to be permeated may be used in the formulation.

Pharmaceutical formulations for parenteral administration, e.g., by bolus injection or continuous infusion, include aqueous solutions of the active compounds in water-soluble form. Additionally, suspensions of the active compounds may be prepared as appropriate oily injection suspensions. Suitable lipophilic solvents or vehicles include fatty oils such as sesame oil, or other organic oils such as soybean, grapefruit or almond oils, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

For oral administration, the agent and/or the compounds (e.g., a polymer conjugate that includes at least one recurring unit selected from Formula (I), (II), (III) and (IV)) can be formulated readily by combining the agent and/or the active compounds with pharmaceutically acceptable carriers well known in the art. Such carriers enable the agent and/or the compounds of the invention to be formulated as tablets, pills, dragees, capsules, liquids, gels, syrups, slurries, suspensions and the like, for oral ingestion by a patient to be treated. Pharmaceutical preparations for oral use can be obtained by combining the agent and/or the active compounds with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the agent and/or the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the agent and/or the compounds (e.g., a polymer conjugate that includes at least one recurring unit selected from Formula (I), (II), (III) and (IV)) are conveniently delivered in the form of an aerosol spray presentation from pressurized packs or a nebulizer, with the use of a suitable propellant, e.g., dichlorodi-fluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the agent and/or the compound and a suitable powder base such as lactose or starch.

Further disclosed herein are various pharmaceutical compositions well known in the pharmaceutical art for uses that include intraocular, intranasal, and intraauricular delivery. Suitable penetrants for these uses are generally known in the art. Pharmaceutical compositions for intraocular delivery include aqueous ophthalmic solutions of the agent and/or the active compounds in water-soluble form, such as eyedrops, or in gellan gum (Shedden et al., Clin. Ther., 23(3):440-50 (2001)) or hydrogels (Mayer et al., Ophthalmologica, 210(2):101-3 (1996)); ophthalmic ointments; ophthalmic suspensions, such as microparticulates, drug-containing small polymeric particles that are suspended in a liquid carrier medium (Joshi, A., J. Ocul. Pharmacol., 10(1):29-45 (1994)), lipid-soluble formulations (Alm et al., Prog. Clin. Biol. Res., 312:447-58 (1989)), and microspheres (Mordenti, Toxicol. Sci., 52(1):101-6 (1999)); and ocular inserts. All of the above-mentioned references, are incorporated herein by reference in their entireties. Such suitable pharmaceutical formulations are most often and preferably formulated to be sterile, isotonic and buffered for stability and comfort. Pharmaceutical compositions for intranasal delivery may also include drops and sprays often prepared to simulate in many respects nasal secretions to ensure maintenance of normal ciliary action. As disclosed in Remington's Pharmaceutical Sciences, 18th Ed., Mack Publishing Co., Easton, Pa. (1990), which is incorporated herein by reference in its entirety, and well-known to those skilled in the art, suitable formulations are most often and preferably isotonic, slightly buffered to maintain a pH of 5.5 to 6.5, and most often and preferably include antimicrobial preservatives and appropriate drug stabilizers. Pharmaceutical formulations for intraauricular delivery include suspensions and ointments for topical application in the ear. Common solvents for such aural formulations include glycerin and water.

The agent and/or the compounds (e.g., a polymer conjugate that includes at least one recurring unit selected from Formula (I), (II), (III) and (IV)) may also be formulated in rectal compositions such as suppositories or retention enemas, e.g., containing conventional suppository bases such as cocoa butter or other glycerides.

In addition to the formulations described previously, the agent and/or the compounds (e.g., a polymer conjugate that includes at least one recurring unit selected from Formula (I), (II), (III) and (IV)) may also be formulated as a depot preparation. Such long acting formulations may be administered by implantation (for example subcutaneously or intramuscularly) or by intramuscular injection. Thus, for example, the agent and/or the compounds may be formulated with suitable polymeric or hydrophobic materials (for example as an emulsion in an acceptable oil) or ion exchange resins, or as sparingly soluble derivatives, for example, as a sparingly soluble salt.

For hydrophobic agent or compounds, a suitable pharmaceutical carrier may be a cosolvent system comprising benzyl alcohol, a nonpolar surfactant, a water-miscible organic polymer, and an aqueous phase. A common cosolvent system used is the VPD co-solvent system, which is a solution of 3% w/v benzyl alcohol, 8% w/v of the nonpolar surfactant Polysorbate 80™, and 65% w/v polyethylene glycol 300, made up to volume in absolute ethanol. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of POLYSORBATE 80™; the fraction size of polyethylene glycol may be varied; other biocompatible polymers may replace polyethylene glycol, e.g., polyvinyl pyrrolidone; and other sugars or polysaccharides may substitute for dextrose.

Alternatively, other delivery systems for hydrophobic pharmaceutical agent or compounds may be employed. Liposomes and emulsions are well known examples of delivery vehicles or carriers for hydrophobic drugs. Certain organic solvents such as dimethylsulfoxide also may be employed, although usually at the cost of greater toxicity. Additionally, the agent and/or the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the agent and/or the compounds for a few hours or weeks up to over 100 days. Depending on the chemical nature and the biological stability of the therapeutic reagent, additional strategies for protein stabilization may be employed.

Agents intended to be administered intracellularly may be administered using techniques well known to those of ordinary skill in the art. For example, such agents may be encapsulated into liposomes. All molecules present in an aqueous solution at the time of liposome formation are incorporated into the aqueous interior. The liposomal contents are both protected from the external micro-environment and, because liposomes fuse with cell membranes, are efficiently delivered into the cell cytoplasm. The liposome may be coated with a tissue-specific antibody. The liposomes will be targeted to and taken up selectively by the desired organ. Alternatively, small hydrophobic organic molecules may be directly administered intracellularly.

Additional therapeutic or diagnostic agents may be incorporated into the pharmaceutical compositions. Alternatively or additionally, pharmaceutical compositions may be combined with other compositions that contain other therapeutic or diagnostic agents.

The agent and/or the compounds (e.g., a polymer conjugate that includes at least one recurring unit selected from Formula (I), (II), (III) and (IV)) or pharmaceutical compositions thereof may be administered to the patient by any suitable means. Non-limiting examples of methods of administration include, among others, (a) administration though oral pathways, which administration includes administration in capsule, tablet, granule, spray, syrup, or other such forms; (b) administration through non-oral pathways such as rectal, vaginal, intraurethral, intraocular, intranasal, or intraauricular, which administration includes administration as an aqueous suspension, an oily preparation or the like or as a drip, spray, suppository, salve, ointment or the like; (c) administration via injection, subcutaneously, intraperitoneally, intravenously, intramuscularly, intradermally, intraorbitally, intracapsularly, intraspinally, intrasternally, or the like, including infusion pump delivery; (d) administration locally such as by injection directly in the renal or cardiac area, e.g., by depot implantation; as well as (e) administration topically; as deemed appropriate by those of skill in the art for bringing the active compound into contact with living tissue.

Pharmaceutical compositions suitable for administration include compositions where the active ingredients are contained in an amount effective to achieve its intended purpose. The effective amount of the compounds disclosed herein required as a dose will depend on the route of administration, the type of animal, including human, being treated, and the physical characteristics of the specific animal under consideration. The dose can be tailored to achieve a desired effect, but will depend on such factors as weight, diet, concurrent medication and other factors which those skilled in the medical arts will recognize. More specifically, an effective amount means an amount of compound effective to prevent, alleviate or ameliorate symptoms of disease or prolong the survival of the subject being treated. Determination of an effective amount is well within the capability of those skilled in the art, especially in light of the detailed disclosure provided herein.

As will be readily apparent to one skilled in the art, the useful in vivo dosage to be administered and the particular mode of administration will vary depending upon the age, weight and mammalian species treated, the particular agent and/or compounds employed, and the specific use for which these agent and/or compounds are employed. The determination of effective dosage levels, that is the dosage levels necessary to achieve the desired result, can be accomplished by one skilled in the art using routine pharmacological methods. Typically, human clinical applications of products are commenced at lower dosage levels, with dosage level being increased until the desired effect is achieved. Alternatively, acceptable in vitro studies can be used to establish useful doses and routes of administration of the compositions identified by the present methods using established pharmacological methods.

In non-human animal studies, applications of potential products are commenced at higher dosage levels, with dosage being decreased until the desired effect is no longer achieved or adverse side effects disappear. The dosage may range broadly, depending upon the desired effects and the therapeutic indication. Typically, dosages may be between about 10 microgram/kilogram (microg/kg) and 100 mg/kg body weight, preferably between about 100 microg/kg and 10 mg/kg body weight. Alternatively dosages may be based and calculated upon the surface area of the patient, as understood by those of skill in the art.

The exact formulation, route of administration and dosage for the pharmaceutical compositions described herein can be chosen by the individual physician in view of the patient's condition. (See e.g., Fingl et al. 1975, in “The Pharmacological Basis of Therapeutics”, which is hereby incorporated herein by reference in its entirety, with particular reference to Ch. 1, p. 1). Typically, the dose range of the composition administered to the patient can be from about 0.5 to 1000 mg/kg of the patient's body weight. The dosage may be a single one or a series of two or more given in the course of one or more days, as is needed by the patient. In instances where human dosages for an agent and/or compounds have been established for at least some condition, the present invention will use those same dosages, or dosages that are between about 0.1% and 500%, more preferably between about 25% and 250% of the established human dosage. Where no human dosage is established, as will be the case for newly-discovered pharmaceutical compositions, a suitable human dosage can be inferred from ED₅₀ or ID₅₀ values, or other appropriate values derived from in vitro or in vivo studies, as qualified by toxicity studies and efficacy studies in animals.

It should be noted that the attending physician would know how to and when to terminate, interrupt, or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the condition to be treated and to the route of administration. The severity of the condition may, for example, be evaluated, in part, by standard prognostic evaluation methods. Further, the dose and perhaps dose frequency, will also vary according to the age, body weight, and response of the individual patient. A program comparable to that discussed above may be used in veterinary medicine.

Although the exact dosage will be determined on a drug-by-drug basis, in most cases, some generalizations regarding the dosage can be made. The daily dosage regimen for an adult human patient may be, for example, an oral dose of between 0.1 mg and 2000 mg of each active ingredient, preferably between 1 mg and 500 mg, e.g. 5 to 200 mg. In other embodiments, an intravenous, subcutaneous, or intramuscular dose of each active ingredient of between 0.01 mg and 100 mg, preferably between 0.1 mg and 60 mg, e.g. 1 to 40 mg is used. In cases of administration of a pharmaceutically acceptable salt, dosages may be calculated as the free base. In some embodiments, the composition is administered 1 to 4 times per day. Alternatively the compositions of the invention may be administered by continuous intravenous infusion, preferably at a dose of each active ingredient up to 1000 mg per day. As will be understood by those of skill in the art, in certain situations it may be necessary to administer the agent and/or the compounds disclosed herein in amounts that exceed, or even far exceed, the above-stated, preferred dosage range in order to effectively and aggressively treat particularly aggressive diseases or infections. In some embodiments, the agent and/or the compounds will be administered for a period of continuous therapy, for example for a week or more, or for months or years.

Dosage amount and interval may be adjusted individually to provide plasma levels of the active moiety which are sufficient to maintain the modulating effects, or minimal effective concentration (MEC). The MEC will vary for each agent and/or compound but can be estimated from in vitro data. Dosages necessary to achieve the MEC will depend on individual characteristics and route of administration. However, HPLC assays or bioassays can be used to determine plasma concentrations.

Dosage intervals can also be determined using MEC value. Compositions should be administered using a regimen which maintains plasma levels above the MEC for 10-90% of the time, preferably between 30-90% and most preferably between 50-90%.

In cases of local administration or selective uptake, the effective local concentration of the drug may not be related to plasma concentration.

The amount of composition administered may be dependent on the subject being treated, on the subject's weight, the severity of the affliction, the manner of administration and the judgment of the prescribing physician.

The agent and/or compounds disclosed herein (e.g., a polymer conjugate that includes at least one recurring unit selected from Formula (I), (II), (III) and (IV)) can be evaluated for efficacy and toxicity using known methods. For example, the toxicology of a particular agent and/or compound, or of a subset of the agent and/or the compounds, sharing certain chemical moieties, may be established by determining in vitro toxicity towards a cell line, such as a mammalian, and preferably human, cell line. The results of such studies are often predictive of toxicity in animals, such as mammals, or more specifically, humans. Alternatively, the toxicity of particular agent and/or compounds in an animal model, such as mice, rats, rabbits, or monkeys, may be determined using known methods. The efficacy of a particular agent and/or compound may be established using several recognized methods, such as in vitro methods, animal models, or human clinical trials. Recognized in vitro models exist for nearly every class of condition, including but not limited to cancer, cardiovascular disease, and various immune dysfunction. Similarly, acceptable animal models may be used to establish efficacy of chemicals to treat such conditions. When selecting a model to determine efficacy, the skilled artisan can be guided by the state of the art to choose an appropriate model, dose, and route of administration, and regime. Of course, human clinical trials can also be used to determine the efficacy of an agent and/or a compound in humans.

The agent or the composition of the present invention may be supplied in any con-figuration, but from the viewpoint of storage stability, it may be provided in a con-figuration that can be prepared at the time of use, for example in a configuration that allows a doctor and/or a pharmacist, a nurse, another paramedic, etc. to prepare it at the place of treatment or in the vicinity thereof. In this case, the agent or the composition of the present invention is provided as one or more containers containing at least one essential constituent element therefor, and it is prepared prior to use, for example, within 24 hours prior to use, preferably within 3 hours prior to use, and more preferably immediately prior to use. When carrying out the preparation, a reagent, a solvent, preparation equipment, etc. that are normally available in a place of preparation may be used as appropriate.

The present invention therefore also relates to a preparation kit for the agent, the compound or the composition disclosed herein, the kit including one or more containers containing singly or in combination a retinoid, and/or a detectable label, and/or optionally a carrier-constituting substance, and/or optionally other component(s) necessary for the preparation of the agent, the compound or the composition. The present invention also relates to a constituent element necessary for the agent, the compound or the composition provided in the form of such a kit. The kit of the present invention may contain, in addition to the above, instructions, an electronic recording medium such as a CD or DVD related to a process for preparing the agent, the compound or the composition of the present invention, or an administration method, etc. Furthermore, the kit of the present invention may include all of the constituent elements for completing the agent, the compound or the composition of the present invention, but need not always include all of the constituent elements. Therefore, the kit of the present invention need not include a reagent or a solvent that is normally available at a place of medical treatment, an experimental facility, etc. such as, for example, sterile water, physiological saline, or a glucose solution.

The agents, the compounds and the compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions comprising an agent and/or a compound of the invention formulated in a compatible pharmaceutical carrier may also be prepared, placed in an appropriate container, and labeled for treatment of an indicated condition.

The agent, the compound and the composition of the present invention are suited for detecting fibrotic diseases in vivo. Thus, they are suited for non-destructively, preferably non-invasively detecting fibrotic diseases. The term “non-destructively” is meant herein to refer to not destroying tissue subjected to detection. For instance, when the tissue subjected to detection is a liver, this term includes to expose the liver by laparotomy, or to obtain images of the liver surface using an endoscope, but does not comprise to cut the liver and explore its interior. On the other hand, the term “non-invasively” is meant herein to refer to detect the label contained in the imaging agent without intentionally injuring the living body, and typically includes detection from outside of a living body, but also encompasses to detect the label by inserting a detector such as an endoscope or a ultrasonic probe through a natural orifice such as buccal cavity, nasal cavity, anus, urethra, auditory meatus and vagina.

The agent, the compound such as polymers and copolymers comprising at least one recurring unit selected from Formulae (I), (II), (III) and (IV), and the composition of the present invention may have many different uses. In some embodiments, the agent, the compound or the composition described herein may be used to deliver a detectable label to a portion of tissue or a cell. In an embodiment, the agent, the compound or the composition described herein can be used to diagnose a disease or condition such as a disease or condition characterized by fibrosis. In yet one more embodiment, the agent, the compound or the composition described herein can be used to image a portion of tissue or a cell. In some embodiments, the tissue can be fibrous tissue.

In one embodiment, the present invention relates to a method for imaging fibrotic diseases, the method including a step of administering an effective amount of the agent, the compound or the composition of the invention to a subject in need thereof, and a step of detecting the label contained in the administered agent, compound or composition. The effective amount referred to here is, for example, an amount that allows to detect the label on at least one site of the body at at least one time point after administration. It is also preferably an amount that does not cause an adverse effect that exceeds the benefit from administration. Such an amount may be determined as appropriate by an in vitro test using cultured cells or by a test in a model animal such as a mouse, a rat, a dog, or a pig, and such test methods are well known to a person skilled in the art. Examples of such tests are already discussed above. Moreover, the dose of the retinoid, the label and optionally the carrier contained in the imaging agent, the compound or the composition of the invention are known to a person skilled in the art, or may be determined as appropriate by the above-mentioned tests, etc.

As the administration route, there are various routes including both oral and parenteral administrations, and examples thereof include oral, intravenous, intra-muscular, subcutaneous, local, intrapulmonary, intra-airway, intratracheal, intra-bronchial, transnasal, rectal, intraarterial, intraportal, intraventricular, intramedullary, intra-lymph node, intra-lymphatic, intracerebral, intrathecal, intracerebroventricular, transmucosal, percutaneous, intranasal, intraperitoneal and intrauterine routes.

In one embodiment, the present invention provides a method for determining fibrotic disease comprising a step of comparing a signal intensity and/or signal distribution of a label detected from a subject to which the agent, compound or composition of the present invention is administered, with a reference signal intensity and/or a reference signal distribution.

The signal intensity of a label is meant herein to refer to an intensity or a measurement similar thereto of various signal emitted from the label, such as fluorescent signal, luminescent signal, magnetic signal and radioactive signal, and typically is measured by an appropriate detection means, the specific examples thereof are already discussed above. The signal intensity may be those obtained from an entire subject or those obtained from a specific site or region of a subject. The signal intensity may also be an average value or an integrated value with regard to the area or the volume of a site to be measured. In case where a signal intensity changes over time, the signal intensity of the present method may be of a specific time point, or may be integrated for a given time period.

The signal distribution of a label is meant herein to refer to information on the position of a signal emitted from the label in a subject, and it may be bidimensional or tridimensional. By matching the signal distribution with an anatomical relative position of organs or with a structural information of a tissue such as CT image, MRI image or ultrasound image, it is possible to identify from which tissue the signal is emitted. In case where a signal distribution changes over time, the signal distribution of the present method may be of a specific time point, or may be integrated for a given time period.

In the present method, it is possible to evaluate a combination of a signal intensity and a signal distribution. A simultaneous evaluation of both intensity and position of the signal allows a more accurate determination.

A reference signal intensity and/or a reference signal distribution is meant herein to refer to a signal intensity and/or a signal distribution of a label measured in a subject that is known to not have a fibrotic disease, and to which an agent, a compound or a composition of the present invention is administered (also referred to as “negative signal intensity and/or negative signal distribution”), or to a signal intensity and/or a signal distribution of a label measured in a subject that is known to have a fibrotic disease, and to which an agent, a compound or a composition of the present invention is administered (also referred to as “positive signal intensity and/or positive signal distribution”). For instance, if the signal intensity and/or the signal distribution of the label detected in the test subject are/is similar to (e.g., not significantly different from) the negative signal intensity and/or the negative signal distribution, the subject may be determined to be fibrotic disease negative, and if the signal intensity of the subject is significantly higher than the negative signal intensity and/or if the signal distribution of the subject is significantly larger than the negative signal distribution, the subject may be determined to be fibrotic disease positive. Furthermore, if the signal intensity and/or signal distribution of the label detected in the test subject are/is similar to (e.g., not significantly different from) the positive signal intensity and/or the positive signal distribution, the subject may be determined to be fibrotic disease positive.

The present invention further relates to a method of monitoring fibrotic disease comprising a step of comparing a signal intensity and/or a signal distribution of a label detected at a first time point from a subject to which the agent, compound or composition of the present invention is administered, with a signal intensity and/or signal distribution of a label detected at a second time point that is later than the first time point from the subject to which the agent, compound or composition of the present invention is administered. For instance, if the signal intensity at the second time point is lower than that at the first time point, the fibrotic disease may be determined to be improved, and conversely, if the signal intensity at the second time point is higher than that at the second time point, the fibrotic disease may be determined to be worsened. Furthermore, for instance, if the signal distribution at the second time point is narrower than that at the first time point, the fibrotic disease may be determined to be improved, and conversely, if the signal distribution of the second time point is broader than that of the first time point, the fibrotic disease may be determined to be worsened.

The present method may comprise a step of administering an agent, a compound or a composition of the present invention to a subject, and/or a step of detecting the label contained in the administered agent, compound or composition at at least two separate time points, and/or a step of determining a signal intensity and/or signal distribution of the detected label, prior to the above-mentioned step of comparison.

The present invention also relates to a method for determining an effect of a fibrotic disease treatment comprising a step of comparing a signal intensity and/or a signal distribution of a label detected at a first time point from a subject to which the agent, compound or composition of the present invention is administered, with a signal intensity and/or a signal distribution of a label detected at a second time point that is later than the first time point from the subject to which the agent, compound or composition of the present invention is administered, wherein the first time point is before the subject receives the fibrotic disease treatment, and the second time point is after the subject received the fibrotic disease treatment, or alternatively, the first time point is after the subject received a first fibrotic disease treatment, and the second time point is after the subject received a second fibrotic disease treatment that is made after the first fibrotic disease treatment. For instance, if the signal intensity at the second time point is lower than that at the first time point, the fibrotic disease may be determined to be improved, and thus the treatment to be successful, and conversely, if the signal intensity at the second time point is higher than that at the first time point, the fibrotic disease may be determined to be worsened, and thus the treatment to be less successful or unsuccessful. Furthermore, for instance, if the signal distribution at the second time point is narrower than that at the first time point, the fibrotic disease may be determined to be improved, and thus the treatment to be successful, and conversely, if the signal distribution of the second time point is broader than that of the first time point, the fibrotic disease may be determined to be worsened, and thus the treatment to be less successful or unsuccessful.

The present method may comprise a step of treating a fibrotic disease in the subject, and/or a step of administering an agent, a compound or a composition of the present invention to the subject, and/or a step of detecting the label contained in the administered agent, compound or composition at at least two separate time points, and/or a step of determining a signal intensity and/or signal distribution of the detected label, prior to the above-mentioned step of comparison.

In the methods of the present invention disclosed herein, the term “subject” is meant to refer to any living individual, preferably an animal, more preferably a mammal, and yet more preferably a human individual. In the present invention, the subject may be healthy or affected by some disorder, and when imaging, diagnostic, determination or monitoring of a fibrotic disease is intended, it typically means a subject affected or suspected to be affected by a fibrotic disease, and when determination of an effect of fibrotic disease treatment is intended, it typically means a subject which has received or will receive a fibrotic disease treatment.

In the methods of the present invention disclosed herein, the term “treatment” includes all types of medically acceptable preventive and/or therapeutic intervention for the purpose of the cure, temporary remission, or prevention of a disorder. For example, the term “treatment” includes medically acceptable intervention for various purposes, including delaying or stopping the progression of a fibrotic disease, regression or disappearance of lesions, prevention of onset of a fibrotic disease, and prevention of recurrence.

It will be understood by those of skill in the art that numerous and various modifications can be made without departing from the spirit of the present invention. Therefore, it should be clearly understood that the forms of the present invention are illustrative only and not intended to limit the scope of the present invention.

### EXAMPLES

The following examples are provided for the purposes of further describing the embodiments described herein, and do not limit the scope of the invention.

### Example 1

### Synthesis of Retinoid-PGA-DOTA

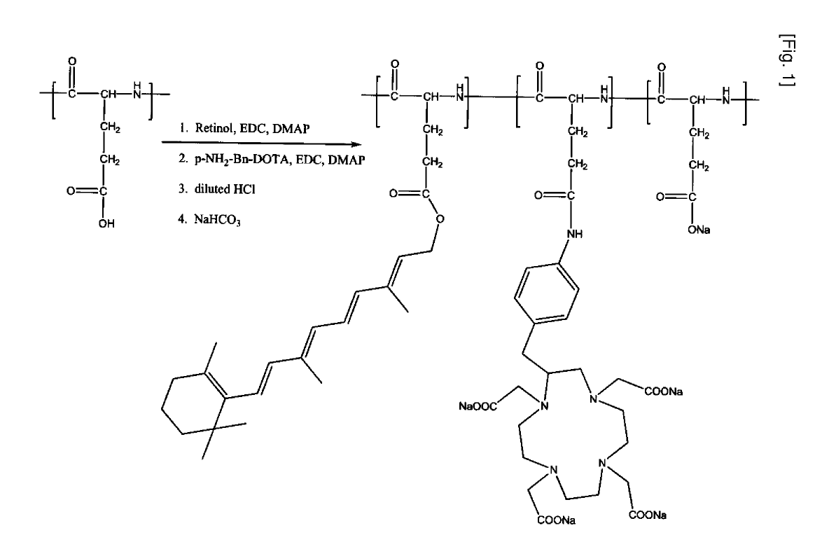

A Retinoid-PGA-DOTA polymer conjugate is prepared according to the general scheme illustrated in FIG. 1 as follows: PGA (150 mg) is dissolved in DMF (15 mL). Retinol (10 mg), EDC (50 mg), and DMAP (50 mg) are added. The mixture is stirred for 24 hours. pNH₂-Bn-DOTA (10 mg), EDC (50 mg), and DMAP (50 mg) are then added. The resulting mixture is stirred for 24 hours. Diluted HCl solution (0.2M) is then added to induce precipitation. The mixture is stirred for 2 minutes and centrifuged at 10,000 rpm for 15 minutes. A solid precipitate is collected, washed with water, and redissolved with sodium bicarbonate solution (0.5 M). The mixture is dialyzed in water for 24 hours. The product, Retinoid-PGA-DOTA polymer conjugate, is freeze-dried. The product's identity is confirmed by ¹H-NMR.

### Example 2

### Synthesis of Retinoid-PGA-DTPA

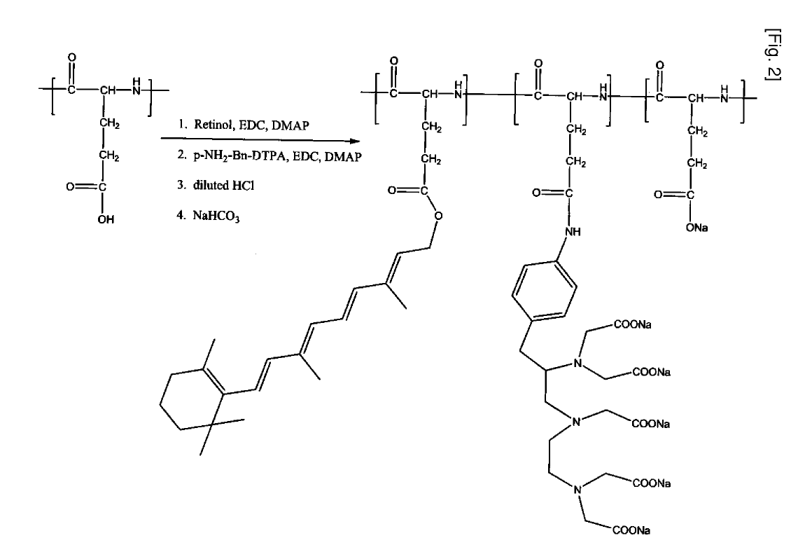

A Retinoid-PGA-DTPA polymer conjugate is prepared according to the general scheme illustrated in FIG. 2 as follows: PGA (150 mg) is dissolved in DMF (15 mL). Retinol (10 mg), EDC (50 mg), and DMAP (50 mg) are added. The mixture is stirred for 24 hours. pNH₂-Bn-DTPA (10 mg), EDC (50 mg), and DMAP (50 mg) are then added. The resulting mixture is stirred for 24 hours. Diluted HCl solution (0.2M) is then added to induce precipitation. The mixture is stirred for 2 minutes and centrifuged at 10,000 rpm for 15 minutes. A solid precipitate is collected, washed with water, and redissolved with sodium bicarbonate solution (0.5 M). The mixture is dialyzed in water for 24 hours. The product, Retinoid-PGA-DTPA polymer conjugate is freeze-dried. The product's identity is confirmed by ¹H-NMR.

### Example 3

### Synthesis of Retinoid-PGGA-DOTA

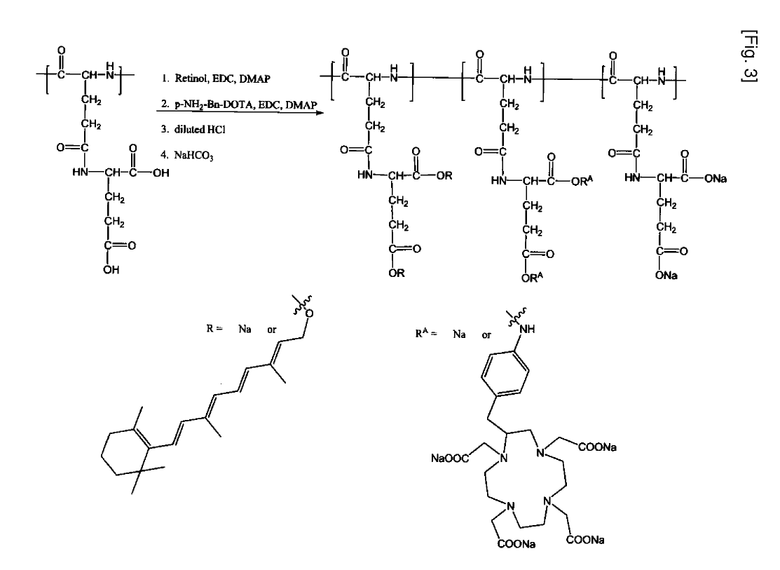

A Retinoid-PGGA-DOTA polymer conjugate is prepared according to the general scheme illustrated in FIG. 3 as follows: Poly(L-gamma-glutamylglutamine) (PGGA, 150 mg) is dissolved in DMF (15 mL). Retinol (10 mg), EDC (50 mg), and DMAP (50 mg) are added. The mixture is stirred for 24 hours. pNH₂-Bn-DOTA (10 mg), EDC (50 mg), and DMAP (50 mg) are then added. The resulting mixture is stirred for 24 hours. Diluted HCl solution (0.2M) is then added to induce precipitation. The mixture is stirred for 2 minutes and centrifuged at 10,000 rpm for 15 minutes. A solid precipitate is collected, washed with water, and redissolved with sodium bicarbonate solution (0.5 M). The mixture is dialyzed in water for 24 hours. The product, Retinoid-PGGA-DOTA polymer conjugate is freeze-dried. The product's identity is confirmed by ¹H-NMR.

### Example 4

### Synthesis of Retinoid-PGGA-DTPA

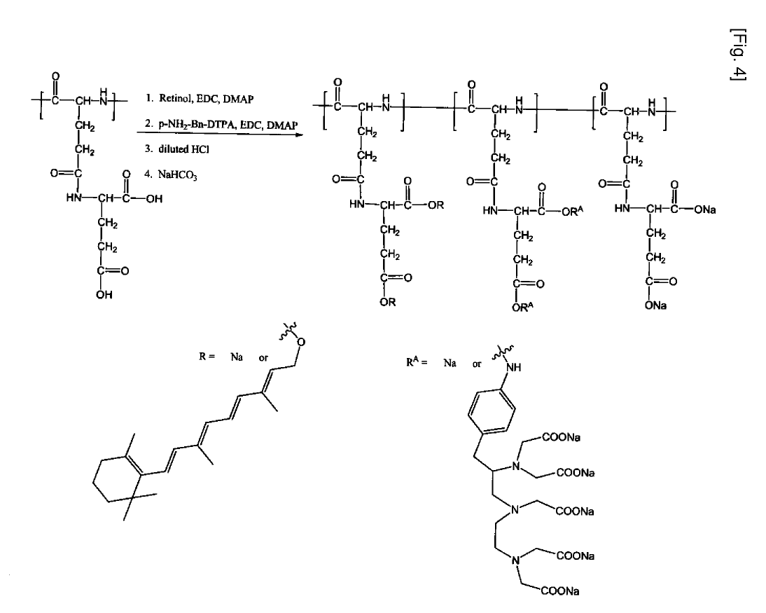

A Retinoid-PGGA-DTPA polymer conjugate is prepared according to the general scheme illustrated in FIG. 4 as follows: Poly(L-gamma-glutamylglutamine) (PGGA, 150 mg) is dissolved in DMF (15 mL). Retinol (15 mg), EDC (50 mg), and DMAP (50 mg) are added. The mixture is stirred for 24 hours. pNH₂-Bn-DTPA(10 mg), EDC (50 mg), and DMAP (50 mg) are then added. The resulting mixture is stirred for 24 hours. Diluted HCl solution (0.2M) is then added to induce precipitation. The mixture is stirred for 2 minutes and centrifuged at 10,000 rpm for 15 minutes. A solid precipitate is collected, washed with water, and redissolved with sodium bicarbonate solution (0.5 M). The mixture is dialyzed in water for 24 hours. The product, Retinoid-PGGA-DTPA polymer conjugate is freeze-dried. The product's identity is confirmed by ¹H-NMR.

### Example 5

### Synthesis of Retinoid-PGA-[(DOTA)Gd(III)]

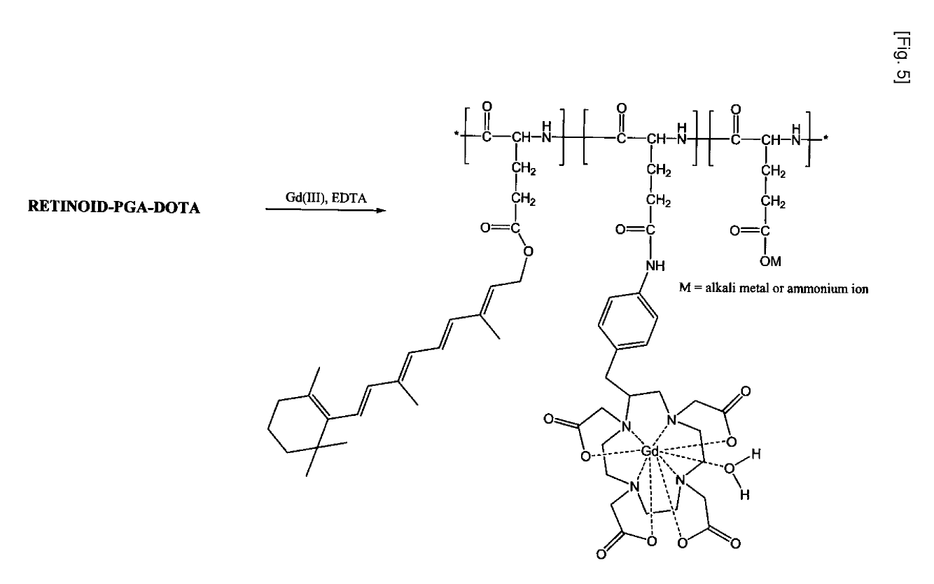

A Retinoid-PGA-[(DOTA)Gd(III)] polymer conjugate is prepared according to the general scheme illustrated in FIG. 5 as follows: Retinoid-PGA-[(DOTA) (45 mg) is dissolved in EDTA buffer (10 mL). A solution of Gd(III) (5 mg) in EDTA (1 mL) is added. The mixture is stirred for 4 hours and poured into sodium bicarbonate solution (50 mL) and dialyzed in water. The product, Retinoid-PGA-[(DOTA)Gd(III)], is lyophilized.

### Example 6

### Synthesis of Retinoid-PGA-[(DTPA)Gd(III)]

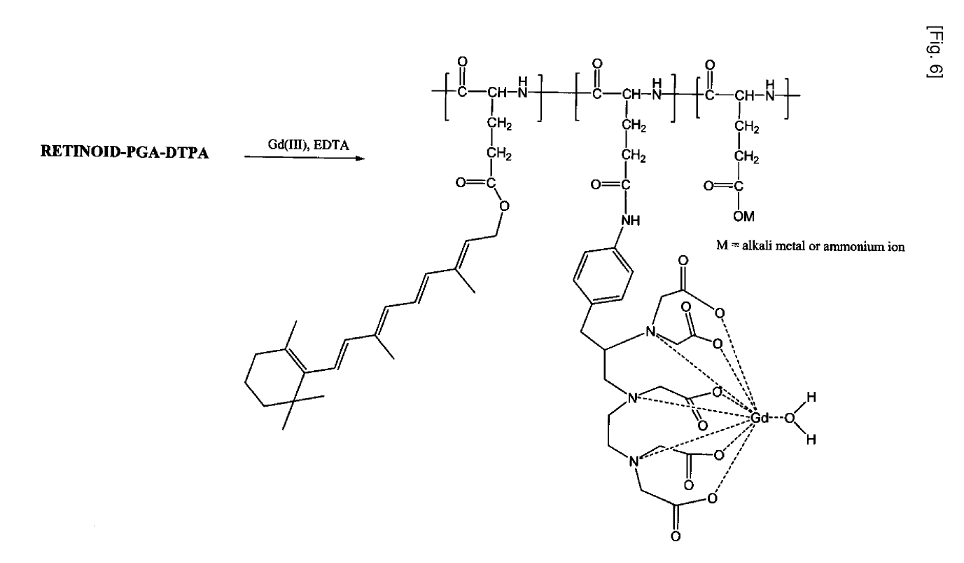

A Retinoid-PGA-[(DTPA)Gd(III)] polymer conjugate is prepared according to the general scheme illustrated in FIG. 6 as follows: Retinoid-PGA-[(DTPA)] (45 mg) is dissolved in EDTA buffer (10 mL). A solution of Gd(III) (5 mg) in EDTA (1 mL) is added. The mixture is stirred for 4 hours and poured into sodium bicarbonate solution (50 mL) and dialyzed in water. The product, Retinoid-PGA-[(DTPA)Gd(III)], is lyophilized.

### Example 7

### Synthesis of Retinoid-PGGA-[(DOTA)Gd(III)]

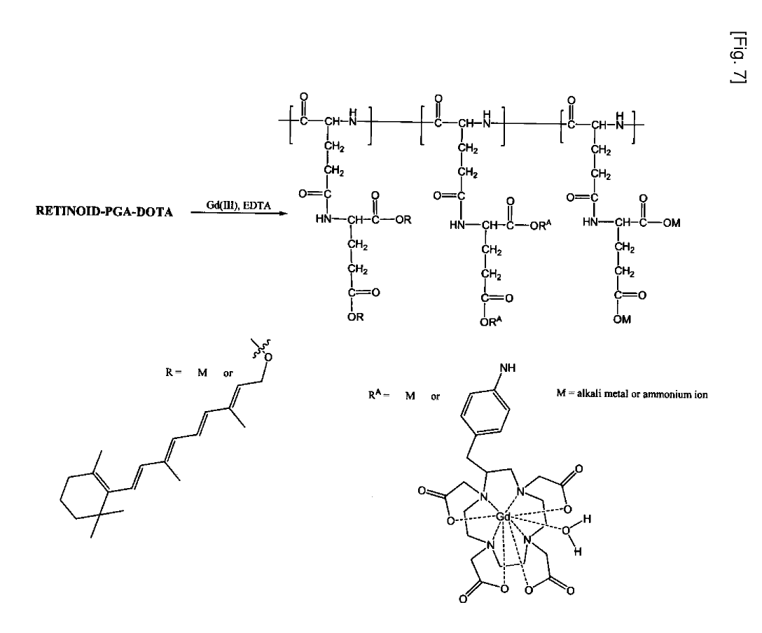

A Retinoid-PGGA-[(DOTA)Gd(III)] polymer conjugate is prepared according to the general scheme illustrated in FIG. 7 as follows: Retinoid-PGA-[(DOTA) (45 mg) is dissolved in EDTA buffers (10 mL). A solution of Gd(III) (5 mg) in EDTA (1 mL) is added. The mixture is stirred for 4 hours and poured into sodium bicarbonate solution (50 mL) and dialyzed in water. The product, Retinoid-PGGA-[(DOTA)Gd(III)], is lyophilized.

### Example 8

### Synthesis of Retinoid-PGGA-[(DPTA)Gd(III)]

A Retinoid-PGGA-[(DTPA)Gd(III)] polymer conjugate was prepared according to the general scheme illustrated in FIG. 8 as follows: Retinoid -PGGA-[(DTPA)] (45 mg) was dissolved in EDTA buffer (10 mL). A solution of Gd(III) (5 mg) in EDTA (1 mL) is added. The mixture was stirred for 4 hours and poured into sodium bicarbonate solution (50 mL) and dialyzed in water. The product, Retinoid —PGGA-[(DTPA)Gd(III)], was lyophilized. The amount of Gd(III) in PGGA-[(DTPA)Gd(III)] was determined by ICP-MS to be 8%.

### Synthesis of PGGA-[(DPTA)Gd(III)]

PGGA-[(DTPA)] (45 mg) was dissolved in EDTA buffer (10 mL). A solution of Gd(III) (5 mg) in EDTA (1 mL) was added. The mixture was stirred for 4 hours and poured into sodium bicarbonate solution (50 mL) and dialyzed in water. The product, PGGA-[(DTPA)Gd(III)], was lyophilized. The amount of Gd(III) in PGGA-[(DTPA)Gd(III)] was determined by inductively coupled plasma-mass spectrometry(ICP-MS) to be 3%.

### Example 9

### Synthesis of Texas Red-Poly(L-glutamic acid)-retinoid (TR-PGA-retinoid)

Poly(L-glutamic acid (PGA, 95.6 mg) was placed into a 50 mL round flask. Anhydrous DMF (15 mL) was added into the flask, and the suspension was stirred for 30 minutes. Retinol (5.5 mg), EDC (12.7 mg), and a trace amount of DMAP were added. The mixture was stirred for 40 hours. Texas Red (1 mg in 1 mL DMF), EDC (300 microL, 5 mg/mL DMF), and HOBt (300 microL, 1 mg/mL DMF) were added to the reaction mixture. The mixture was stirred for 15 hours. The reaction mixture was then poured into 0.2 N HCl aqueous solution (75 mL). The resulting mixture was transferred to centrifuge tubes and centrifuged. The supernatant was discarded. The solid was dissolved in 0.5 N NaHCO₃ aqueous solution (approximately 60 mL). The solution was then dialyzed against deionized water, filtered through a 0.45 micrometer cellular acetate syringe filter and lyophilized. TR-PGA-retinoid (93 mg) was obtained, and characterized by ¹H-NMR and UV-Vis spectroscopy.

### Example 10

### Synthesis of Texas Red-Poly(L-gamma-glutamylglutamine)-Retinoid (TR-PGGA-Retinoid)

Poly(L-gamma-glutamylglutamine) (PGGA, 95.5 mg) was placed into a 50 mL round-bottomed flask. Anhydrous DMF (6 mL) was added into the flask, and the suspension was stirred for 30 minutes. Retinol (5.0 mg), EDC (16.3 mg), and a trace amount of DMAP were added. The mixture was stirred for 40 hours. Texas Red (TR) (1 mg in 1 mL DMF), EDC (300 microL, 5 mg/mL DMF), and HOBt (300 microL, 1 mg/mL DMF) were added to the reaction mixture. The mixture was stirred for 15 hours. The reaction mixture was then poured into 0.2 N HCl aqueous solution (75 mL). The resulting mixture was transferred to centrifuge tubes and centrifuged. The supernatant was discarded. The solid was dissolved in 0.5N NaHCO₃ aqueous solution (approximately 60 mL). The solution was dialyzed against deionized water, filtered through a 0.45-micrometer cellular acetate syringe filter and lyophilized. TR-PGGA-retinoid (91 mg) was obtained, and characterized by ¹H-NMR and UV-Vis spectroscopy.

### Example 11

### Synthesis of Texas Red-Poly(L-glutamic acid)-cholesterol (TR-PGA-cholesterol)

Poly(L-glutamic acid) (PGA, 99.7 mg) was placed into a 50 mL round-bottomed flask. Anhydrous DMF (15 mL) was added to the flask and the suspension was stirred for 30 minutes. Cholesterol (5.9 mg), EDC (10.7 mg), and a trace amount of DMAP were added. The mixture was stirred for 40 hours. Texas Red (1 mg in 1 mL DMF), EDC (300 microL, 5 mg/mL DMF), and HOBt (300 microL, 1 mg/mL DMF) were added to the reaction mixture. The mixture was stirred for 15 hours. The reaction mixture was then poured into 0.2 N HCl aqueous solution (75 mL). The resulting mixture was transferred to centrifuge tubes and centrifuged. The supernatant was discarded. The solid was dissolved in 0.5N NaHCO₃ aqueous solution (approximately 60 mL). The solution was dialyzed against deionized water, filtered through a 0.45 micrometer cellular acetate syringe filter and lyophilized. TR-PGA-cholesterol (90 mg) was obtained, and characterized by ¹H-NMR and UV-Vis spectroscopy.

### Example 12

### HSC-T6 Cells Uptake of Retinoid Compounds

HSC-T6 cells, which express a vitamin A binding protein receptor, were seeded one day prior to procedure in a 96-well plate (100 microL culture medium per well). TR-PGA-retinoid, TR-PGGA-retinoid and TR-PGA-cholesterol, as prepared in Examples 9-11, were dissolved in water to make approximately 2-4 mg/mL stock solutions. The solutions were diluted with culture medium and incubated for 15 minutes at room temperature. 15 microL was added to the cells. After incubating the cells in solution, the culture medium was removed. Cells were washed once with DPBS and fresh culture medium was added (100 microL culture medium per well). Absorbance (excitation and emission wavelengths were 560 nm and 590 nm, respectively) was read by a BioTek FLx800 96-well plate fluorescence reader and recorded. The results are shown in FIG. 8.

FIG. 8 compares the cell uptake of Texas Red-non-cationic polymeric carrier-retinoid with the cell uptake of Texas Red-non-cationic polymeric carrier-cholesterol. Greater absorbance indicates greater optical density and greater cell uptake. Thus, FIG. 8 shows that the retinoid compositions resulted in greater cellular uptake than the cholesterol composition.

### Example 13

### Magnetic Resonance Imaging

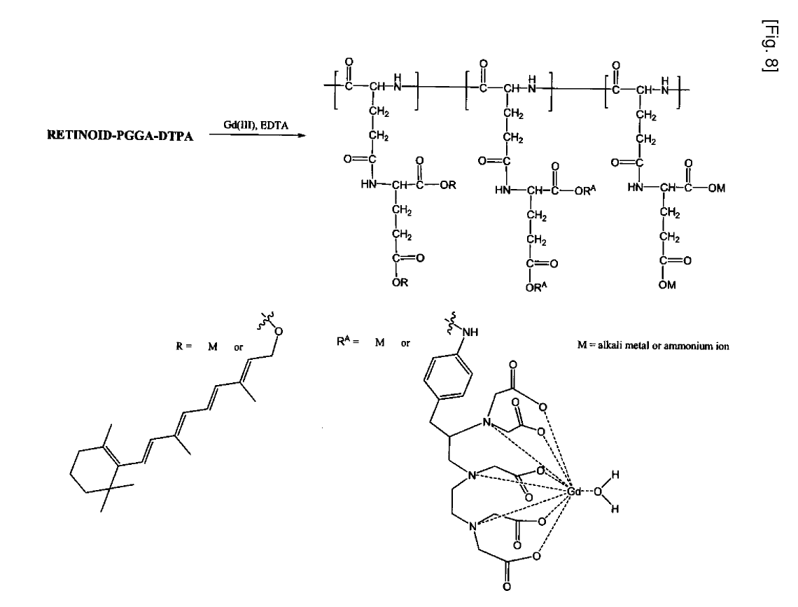

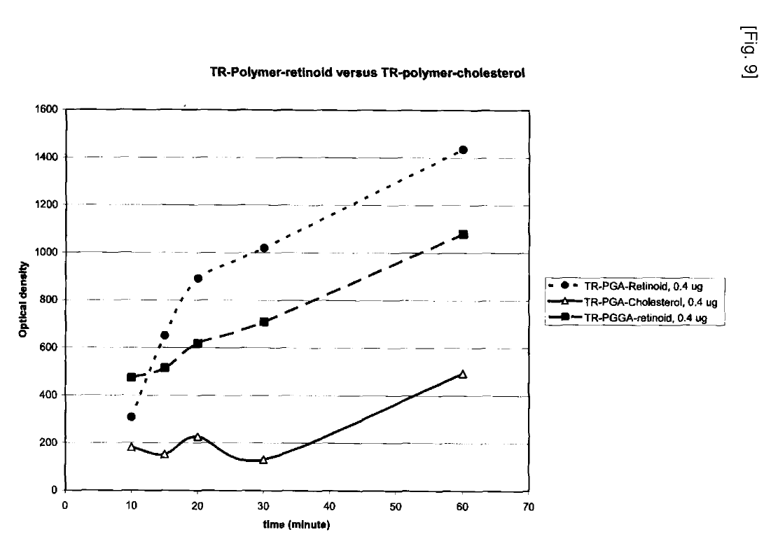

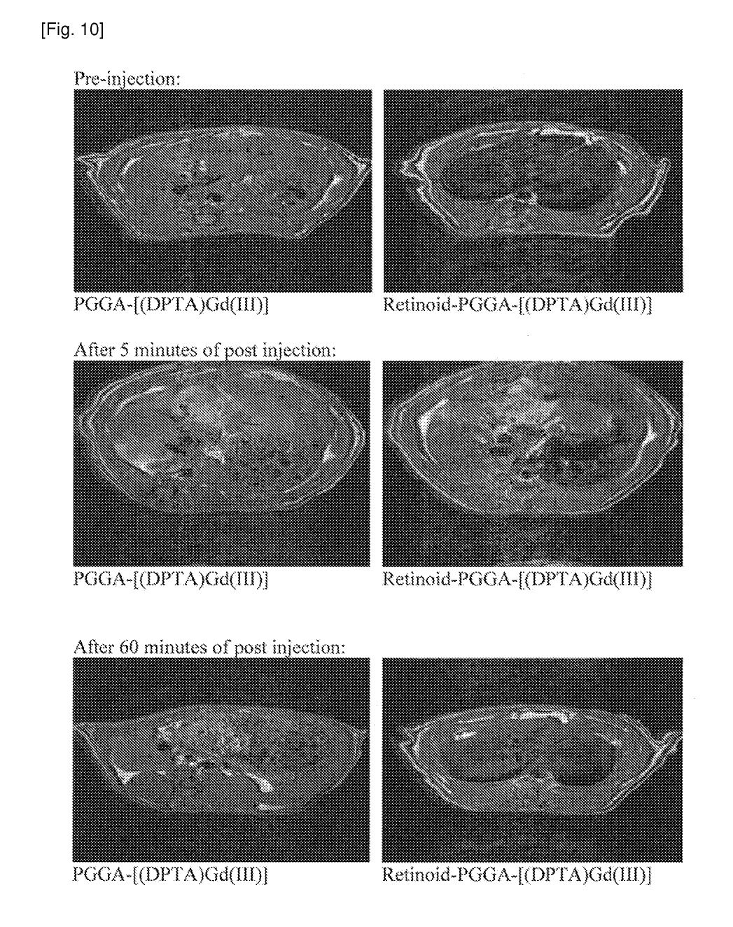

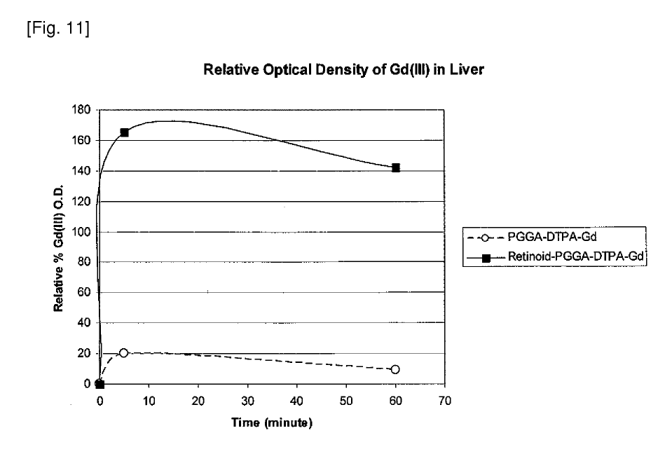

Images of mice are acquired on a GE 3T MR scanner using a knee coil pre- and post-contrast. The following imaging parameters are TE: minful, TR=250 ms, FOV: 8 and 24 slices/slab, and 1.0 mm coronal slice thickness. The dose of injection of the test compounds, Retinoid-PGGA-[(DPTA)Gd(III)] and PGGA-[(DPTA)Gd(III)] obtained as described in Example 8, are 0.05 mmol metal ion/kg to liver fibrosis DMA rat models (n=3 for each time point for each compound). The compounds were injected via a tail vein into anesthetized mice and images are acquired at pre-injection and at 5 minutes, 15 minutes, and 60 minutes post-injection of the contrast agents (see FIG. 9). The average relative optical density of Gd(III) was obtained and showed as in FIG. 10.

### Example 14

### In vivo Imaging of cirrhosis model mouse

A model mouse having carbon-tetrachloride-induced cirrhosis (hereinafter, also referred to as “cirrhotic mouse”) and a normal mouse were used for non-invasive observation of focuses from outside the organism.

As a cirrhotic mouse, a 4-week-old C57BL/6J male mouse (Charles River) which was intraperitoneally injected with CCl₄ (1 microL/g body weight) diluted with olive oil in 1:10, twice a week for 28 weeks. As a normal mouse (control group), a 20-week-old C57BL/6J male mouse was used.

From 2 weeks before the observation, the mice were bred as usual with alfalfa-free diet in order to reduce the effect of autonomous fluorescence derived from diet in the gastrointestinal tract. To reduce the degree of a signal decrease due to skin hair, the hair of the abdomen and back was removed.

As a fluorescent probe for detecting signals inside an organism, Cy™5.5-labeled scramble siRNA (sense: 5′-Cy™5.5-CUUACGCUGAGUACUUCGATT-3′ (SEQ ID NO: 1), antisense: 5′-Cy™5.5-UCGAAGUACUCAGCGUAAGTT-3′ (SEQ ID NO: 2); hereinafter also referred to as “siRNA scr-Cy™5.5”) was used. As a carrier, Lipotrust SR (Hokkaido System Science Co., Ltd.) (hereinafter, also referred to as “Liposome”) was used. As a pre-mixture solution, 100 mM of vitamin A (retinol (Sigma), hereinafter also referred to as VA; dissolved in dimethylsulfoxide), 1 mM of Lipotrust SR (dissolved in nuclease-free water), and siRNA scr-Cy™5.5 diluted to 10 microg/microL in nuclease-free water were prepared. First, Lipotrust SR and VA were mixed in 1:1 (mol/mol), vortex-stirred for 15 s, then left to stand at room temperature under light shielding for 5 min to form a complex. To this complex, 10 microg/microL of siRNA scr-Cy™5.5 was added and mixed gently, to give an imaging agent (VA-Lip-Cy) of the present invention. The composition of the obtained imaging agent was, relative to 100 microL of the imaging agent, 100 nmol (28.6 mg) of retinol, 100 nmol (62.6 mg) of Liposome-forming cationic lipid, and 10 microg of Cy™5.5-labeled SiRNA. This imaging agent is that in which at least a part of the retinoid is exposed outside the imaging agent before it reaches target cells at the latest. An imaging agent without VA (Lip-Cy) was produced similarly. These imaging agents were slowly administered to mice having 30-g body weight via the tail vein under isoflurane gas anesthesia, at an amount of 100 microL per mouse.

Before the administration, and 5 min to 90 min after the administration, fluorescent sites of the mouse were observed over time using IVIS Imaging System (XENOGEN, IVIS®200), and fluorescent signals were quantified. For quantification, a physical quantity (photon/sec; hereinafter also expressed as p/s) based on the Tissue-Diffusion Model theory was measured instead of luminescence intensity (counts), and the measurements were numerically expressed by average radiance (Avg Radiance, p/s/cm²/sr) and plotted in a graph after the correction of excitation light source (expressed by average efficacy (Avg Efficacy)). Here, “p/s/cm²/sr” is an abbreviation of “photons per second per square centimeter per steradian,” where steradian is a unit of solid angle.

At 90 min after the administration, the mouse was sacrificed and the liver was removed, fixed with 4% paraformaldehyde, and paraffin-embedded to make a thin-section sample. The sample was stained with alpha-SMA-FITC (Sigma, F3777) to confirm intracellular localization of siRNA scr-Cy™5.5 signals. In addition, the ratio of an area positive for both Cy™5.5 and FITC relative to the total Cy™5.5-positive area (alpha-SMA merge/Cy™5.5 positive area), and the ratio of the number of cells positive for both Cy™5.5 and FITC relative to the total number of Cy™5.5-positive cells (alpha-SMA merge/Cy™5.5 positive cells) were analyzed.

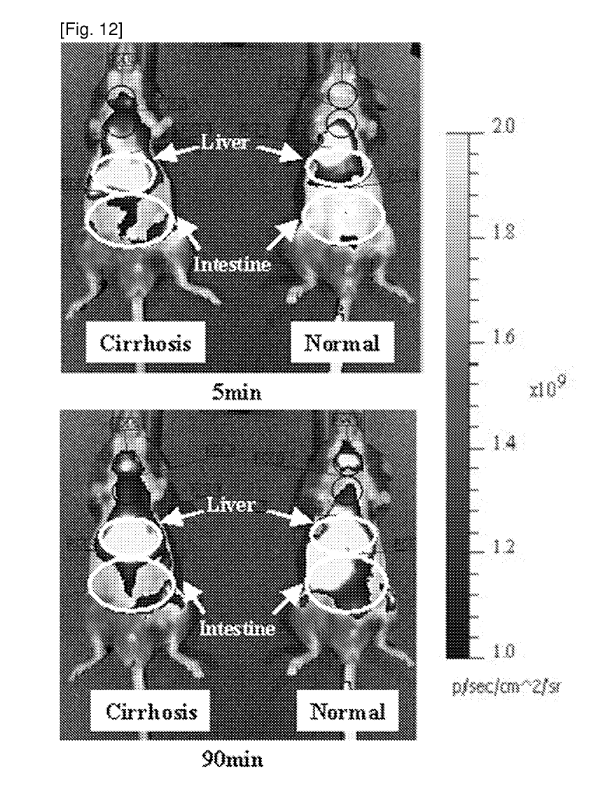

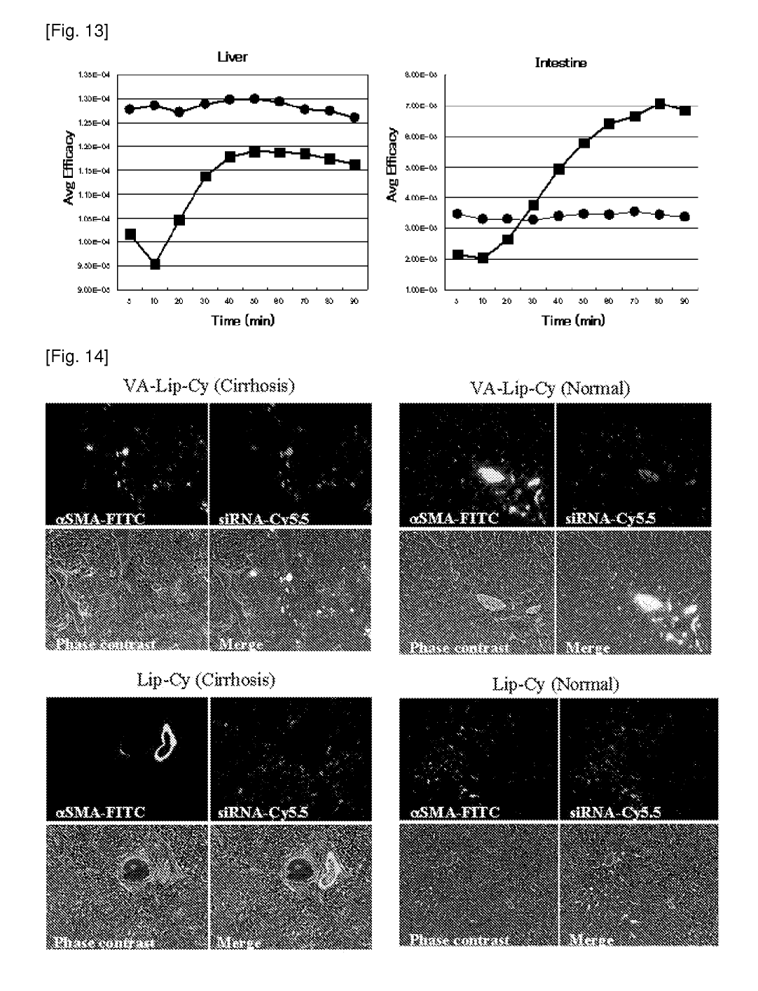

Both in the cirrhotic mouse administered with VA-Lip-Cy (Cirrhosis) and in the normal mouse (Normal), signals were observed in the liver (Liver) from 5 min after the start of the administration, but the signals were significantly stronger in the cirrhotic mouse than in the normal mouse. In contrast, signals in the intestinal tract (Intestine) in the cirrhotic mouse showed almost no change, whereas those in the normal mouse showed a gradual increase from 30 min after the start of the administration (FIGS. 12 and 13).

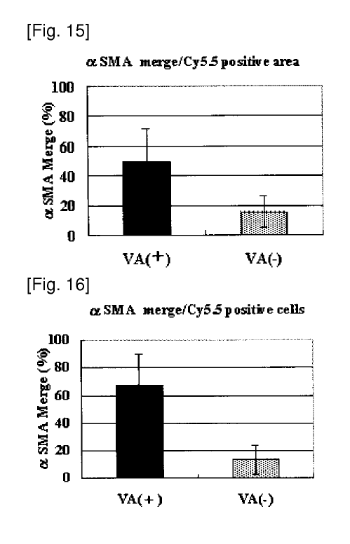

From the results of staining of liver samples, it can be seen that the number of cells positive for both alpha-SMA (FITC) and SiRNA (Cy™5.5) in the cirrhosis mouse administered with VA-Lip-Cy (VA(+)) was significantly larger than that in the cirrhotic mouse administered with Lip-Cy (VA(−)) or in the normal mouse administered with VA-Lip-Cy (FIGS. 14-16).

These results show that, in a cirrhotic mouse, the imaging agent of the present invention specifically label alpha-SMA-positive activated stellate cells and stays at the fibrotic focus, whereas in a normal mouse, the imaging agent of the present invention does not stay in the liver but transfers to the intestinal tract. Accordingly, by the observation and quantification of such a label from outside an organism, presence/absence of a fibrotic disease and its severity can be determined or diagnosed non-invasively and easily. Moreover, non-invasive and temporal observation of a single individual becomes possible, thereby enabling evaluation of therapy with high accuracy.

1. An imaging agent of a cell and/or tissue characterized by fibrosis comprising a retinoid and a detectable label.

2. The imaging agent of claim 1, wherein the retinoid comprises retinol.

3. The imaging agent of claim 1, wherein the imaging agent is for in vivo imaging.

4. The imaging agent of claim 1, wherein the imaging agent is for fibrotic disease imaging.

5. The imaging agent of claim 1, comprising a polymer conjugate comprising at least one recurring unit selected from Formulae (I), (II), (III) and (IV): 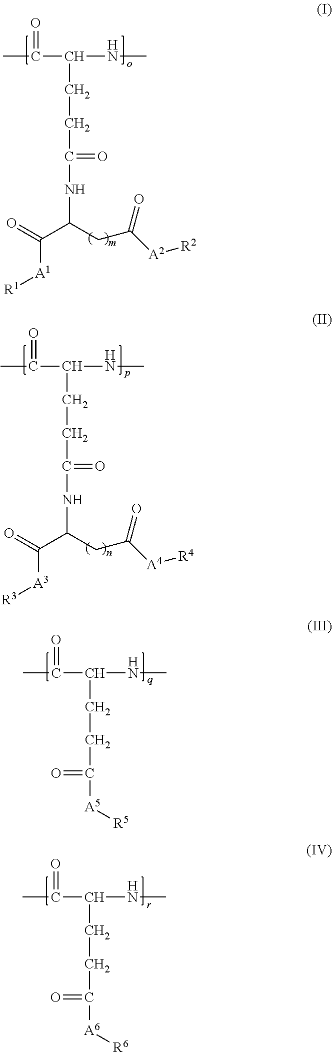
wherein:
m is independently 1 or 2;
n is independently 1 or 2;
A¹ and A² are each independently oxygen or NR⁷;
A³ and A⁴ are each independently oxygen or NR⁸;
A⁵ and A⁶ are each independently oxygen or NR⁹;
R¹, R², R³, R⁴, R⁵, and R⁶ are each independently selected from the group consisting of optionally substituted C₁₋₁₀ alkyl, optionally substituted C₆₋₂₀ aryl, ammonium, alkali metal, a retinoid and a group that comprises a detectable label;
R⁷, R⁸ and R⁹ are each independently hydrogen or C₁₋₄ alkyl;
o, p, q, and r are each independently 0, 1 or greater, wherein the sum of o, p, q, and r is 2 or greater; and
provided that at least one of R¹, R², R³, R⁴, R⁵, and R⁶ is a group that comprises a detectable label and at least one of R¹, R², R³, R⁴, R⁵, and R⁶ is a retinoid.

6. A polymer conjugate comprising at least one recurring unit selected from Formulae (I), (II), (III) and (IV): 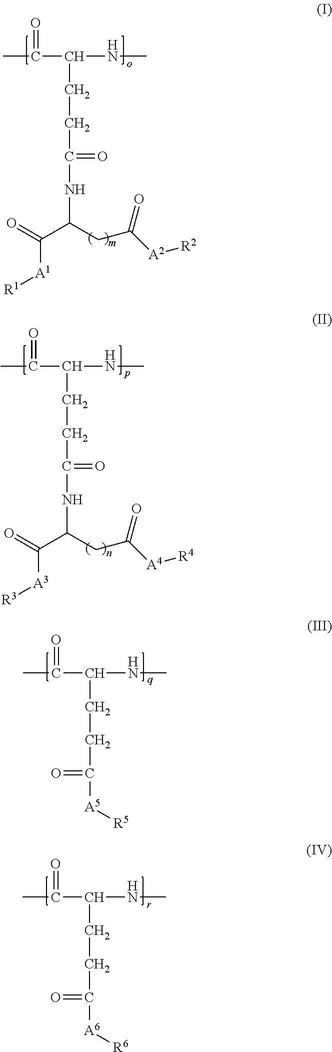
wherein:
m is independently 1 or 2;
n is independently 1 or 2;
A¹ and A² are each independently oxygen or NR⁷;
A³ and A⁴ are each independently oxygen or NR⁸;
A⁵ and A⁶ are each independently oxygen or NR⁹;
R¹, R², R³, R⁴, R⁵, and R⁶ are each independently selected from the group consisting of optionally substituted C₁₋₁₀ alkyl, optionally substituted C₆₋₂₀ aryl, ammonium, alkali metal, a retinoid and a group that comprises a detectable label;
R⁷, R⁸ and R⁹ are each independently hydrogen or C₁₋₄ alkyl; o, p, q, and r are each independently 0, 1 or greater, wherein the sum of o, p, q, and r is 2 or greater; and
provided that at least one of R¹, R², R³, R⁴, R⁵, and R⁶ is a group that comprises a detectable label and at least one of R¹, R², R³, R⁴, R⁵, and R⁶ is a retinoid.

7. The polymer conjugate of claim 6, wherein the polymer further comprises at least one recurring unit of Formula (V): 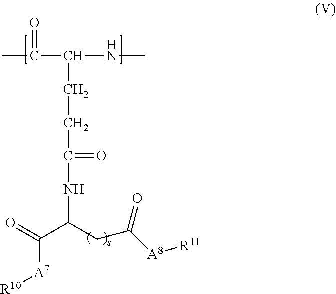
wherein:
s is independently 1 or 2;
A⁷ and A⁸ are each independently oxygen or NR¹²;
R¹² is hydrogen or C₁₋₄ alkyl;
R¹⁰ and R¹¹ are each independently selected from the group consisting of optionally substituted C₁₋₁₀ alkyl, optionally substituted C₆₋₂₀ aryl, ammonium and alkali metal.

8. The polymer conjugate of claim 6, wherein the polymer further comprises at least one recurring unit of Formula (VI): 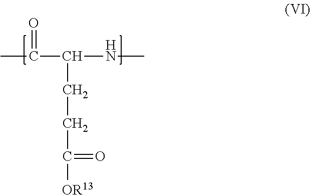
wherein R¹³ is hydrogen, ammonium, or an alkali metal.

9. The polymer conjugate of claim 6, wherein the detectable label comprises a metal selected from the group consisting of Gd(III), Yttrium-88 and Indium-111.

10. The polymer conjugate of claim 6, wherein the detectable label comprises a ligand selected from the group consisting of: diethylenetriaminepentacetic acid (DTPA), tetraazacyclododecane-1,4,7,10-tetraacetic acid (DOTA), (1,2-ethanediyldinitrilo)tetraacetate (EDTA), ethylenediamine, 2,2′-bipyridine (bipy), 1,10-phenanthroline (phen), 1,2-bis(diphenylphosphino)ethane (DPPE), 2,4-pentanedione (acac), and ethanedioate (ox).

11. The polymer conjugate of claim 6, wherein the detectable label comprises a ligand selected from the group consisting of: diethylenetriaminepentacetic acid (DTPA) and tetraazacyclododecane-1,4,7,10-tetraacetic acid (DOTA).

12. The polymer conjugate of claim 6, wherein the detectable label is a paramagnetic metal chelate.

13. The polymer conjugate of claim 12, wherein the paramagnetic metal chelate comprises

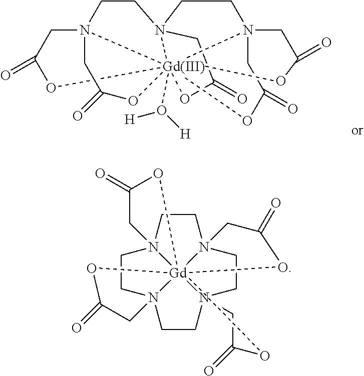

14. The polymer conjugate of claim 6, wherein the detectable label is a dye.

15. The polymer conjugate of claim 14, wherein the dye comprises Texas Red.

16. The polymer conjugate of claim 6, wherein m is 1.

17. The polymer conjugate of claim 6, wherein m is 2.

18. The polymer conjugate of claim 6, wherein n is 1.

19. The polymer conjugate of claim 6, wherein n is 2.

20. The polymer conjugate of claim 7, wherein s is 1.

21. The polymer conjugate of claim 7, wherein s is 2.

22. A method of making the polymer conjugate of claim 6 comprising dissolving or partially dissolving a polymeric reactant comprising at least one of a recurring unit of Formula (VII) and a recurring unit of Formula (VIII) in a solvent to form a dissolved or partially dissolved polymeric reactant; 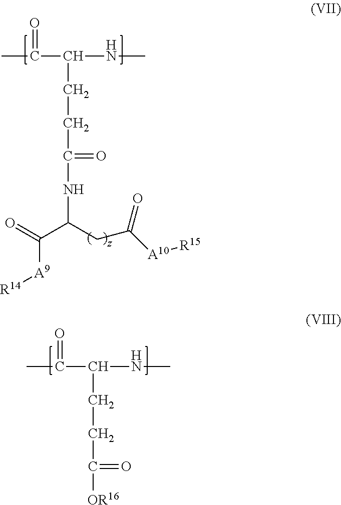
wherein:
z is 1 or 2;
A⁹ and A¹⁰ are oxygen; and
R¹⁴, R¹⁵, and R¹⁶ are each independently selected from the group consisting of hydrogen, ammonium, and an alkali metal; and
reacting the dissolved or partially dissolved polymeric reactant with a second reactant, wherein the second reactant comprises the group comprising the detectable label or the retinoid; and
adding a third reactant, wherein the third reactant comprises the group comprising the detectable label, a ligand or the retinoid; provided that if the second reactant comprises the group comprising the detectable label or the ligand then the third reactant comprises the retinoid and if the second reactant comprises the retinoid then the third reactant comprises the group that comprises the detectable label or the ligand.

23. The method of claim 22, wherein the second reactant comprises the retinoid.

24. The method of claim 22, wherein the third reactant comprises the group that comprises the detectable label.

25. The method of claim 22, wherein the third reactant comprises the ligand.

26. The method of claim 25, wherein the ligand is selected from the group consisting of: diethylenetriaminepentacetic acid (DTPA), tetraazacyclododecane-1,4,7,10-tetraacetic acid (DOTA), (1,2-ethanediyldinitrilo)tetraacetate (EDTA), ethylenediamine, 2,2′-bipyridine (bipy), 1,10-phenanthroline (phen), 1,2-bis(diphenylphosphino)ethane (DPPE), 2,4-pentanedione (acac), and ethanedioate (ox).

27. The method of claim 22, further comprising adding a fourth reactant, wherein the fourth reactant comprises a metal.

28. The method of claim 27, wherein the metal is selected from the group consisting of Gd(III), Yttrium-88 and Indium-111.

29. A diagnostic agent of fibrotic disease comprising the imaging agent of claim 1.

30. A composition comprising the imaging agent of claim 1, and at least one selected from a pharmaceutically acceptable excipient, a carrier, and a diluent.

31. A method of delivering a detectable label to a portion of tissue comprising contacting the portion of tissue or a cell with at least one imaging agent of claim 1.

32. A method of imaging a portion of tissue comprising contacting the portion of tissue or a cell with at least one imaging agent of claim 1.

33. A method of diagnosing a disease or condition comprising contacting a portion of tissue or a cell with at least one polymer conjugate of claim 6.

34. The method of claim 31, wherein the tissue is fibrous tissue.

35. A method for imaging a fibrotic disease comprising a step of administering an effective amount of the imaging agent of claim 1 to a subject in need thereof, and a step of detecting the label contained in the administered imaging agent, polymer conjugate or composition.

36. A method for determining fibrotic disease comprising a step of comparing a signal intensity and/or a signal distribution of a label detected from a subject to which the imaging agent of claim 1 is administered, with a reference signal intensity and/or a reference signal distribution.

37. A method of monitoring fibrotic disease comprising a step of comparing a signal intensity and/or a signal distribution of a label detected at a first time point from a subject to which the imaging agent of claim 1 is administered, with a signal intensity and/or a signal distribution of a label detected at a second time point that is later than the first time point from said subject.

38. A method for determining an effect of a fibrotic disease treatment comprising a step of comparing a signal intensity and/or a signal distribution of a label detected at a first time point from a subject to which the imaging agent of claim 1 is administered, with a signal intensity and/or a signal distribution of a label detected at a second time point that is later than the first time point from said subject, wherein the first time point is before the subject receives the fibrotic disease treatment, and the second time point is after the subject has received the fibrotic disease treatment, or alternatively, the first time point is after the subject received a first fibrotic disease treatment, and the second time point is after the subject received a second fibrotic disease treatment that is made after the first fibrotic disease treatment.
